(19) United States
(12) Patent Application Publication
(10) Pub. No.: US 2004/0137631 A1
(43) Pub. Date: Jul. 15, 2004

(54) Processes and vectors for plastid transformation

(76) Inventors: Stefan Herz, Munich (DE); Sebastian Klaus, Grafelfing (DE); Christian Eibl, Ismaning (DE); Stefan Muhlbauer, Freising (DE); Hans-Ulrich Koop, Munich (DE); Yuri Gleba, Halle (DE)

Correspondence Address: MYERS BIGEL SIBLEY & SAJOVEC, PO BOX 37428, RALEIGH, NC, 27627, US

(21) Appl. No.: 10/466,369

(22) PCT Filed: Jan. 14, 2002
(86) PCT No.: PCT/EP02/00301

(30) Foreign Application Priority Data
Jan. 12, 2001 (DE) 10101276.4

(51) Int. Cl. A01H 1/00; C12N 15/82

(52) U.S. Cl. 435/468; 800/278

(57) ABSTRACT

This invention discloses a novel process and vector therefore for producing a multicellular plant or plant cells having stably transformed plastids. This process comprises transforming plastids or plant cells via homologous recombination with a DNA molecule enabling DNA modification, whereby said DNA molecule comprises a fragment of a gene requiring for expression a sequence element of the host plastid. The invention also includes the use of novel selection inhibitors for plastic transformation and the possibility of performing multiple rounds of transformation without accumulation of resistance genes.

### FIELD OF INVENTION

The present invention relates to plant biotechnology in general and to novel transformation vectors and selection protocols for plastid transformation. The invention further relates to a novel process of multiple transfomations and to novel selection protocols.

### BACKGROUND OF THE INVENTION

Plastid transformation makes use of the enormous copy number advantage—more than 10000 copies of the plastome per cell may be present—over nuclear-expressed genes to permit expression levels that may exceed 10% of the total soluble plant protein. In addition, plastid transformation is desirable because plastid-encoded traits are not pollen transmissible; hence, potential risks of inadvertent transgene escape to wild relatives of transgenic plants are largely reduced. Conventional plastid transformation technology is described in Heifetz, 2000 and Koop et al., 1996.

Conventional plastid transformation vectors usually need to serve at least two purposes: (1) introduction of one or more desired foreign genes to be expressed by the genetic machinery of the plastids, and (2) selection of cells containing transformed plastomes by inhibitor selection or by screening for a detectable phenotype. Plastid transformation vectors usually contain at least two complete gene cassettes, each consisting of four operably linked elements: a promoter sequence, a 5′ untranslated region, a coding region, and a 3′ untranslated region. As an exception to this rule, a vector has been suggested which has only in one of its gene cassettes a promoter element (Staub & Maliga, 1995). Such approach has, however, not been applied to a selection gene cassette.

Selection is achieved either by replacing a complete resident plastid gene by a mutant gene, which confers resistance to selection inhibitors (U.S. Pat. No. 5,451,513) or by introducing a complete expression cassette, which leads to enzymatic inactivation of an inhibitor (U.S. Pat. No. 5,877,402).

In addition to the two or more gene cassettes, transformation vectors contain flanking regions of the insertion site, which are necessary for the introduction of engineered sequences into the plastome by homologous recombination.

In the above conventional plastid transformation method, novel sequences, i.e. the non-plastid gene expression cassette supplying enzymatic inhibitor inactivation, and the expression cassettes for desired additional genes, are usually targeted to non-transcribed regions in order not to interfere with plastid gene functions (U.S. Pat. Nos. 5,877,402, 5,932,479, WO9910513, WO9855595, WO9946394, WO9706250, WO0007431). If they are targeted to a transcribed region, interference is accepted.

Conventional vectors for plastid transformation give rise to transformants with a certain instability. It has been observed that loss of a foreign gene is frequently encountered. This has been an intrinsic problem of conventional plastid transformation procedures.

### SUMMARY OF THE INVENTION

It is the problem of this invention to provide an alternative and highly versatile process for plastid transformation and vectors therefore, which gives rise to transformants with increased stability.

It is another problem of the invention to provide a process of plastid transformation with increased biological safety.

This problem is solved by the process for producing a multicellular plant or plant cells having stably transformed plastids comprising the following steps:

a) transforming plastids of said plant or plant cells via homologous recombination with at least one DNA molecule for enabling DNA modification, whereby said DNA molecule comprises a fragment of a gene requiring for expression in a transformed plastid a sequence element of the host plastid not present in said DNA molecule;

b) subjecting said plastids to growth conditions which favour multiplication or allow for identification of plastids having said DNA modification; and

c) selecting or identifying plastids that are functional and contain information encoded in said DNA molecule;

thereby giving rise to functional cells and multicellular plants with stably transformed plastids.

Preferred embodiments are defined in the subclaims.

Further, this invention provides vectors for the above processes.

It has been surprisingly found that it is not required to use vectors containing a complete gene expression cassette for plastid transformation and functional transgene expression in plastids. This has the advantage that the transformed sequences do not have to come equipped with all sequences, notably also control sequences, for autonomous function. Rather, they can rely on a sequence element, notably a control sequence element, of a plastid gene or operon. It has been surprisingly found that such a novel transformation process leads to much increased stability of transformation. The exact mechanism of this increased stability has not yet been investigated. It is comtemplated that it is due to a reduced propensity for homologous recombination due to a reduction of homologous sequences. The simplified vectors are less constrained and open up new avenues of transformation not available with the conventional processes.

The DNA modification can be sequence insertion, replacement and/or deletion.

The gene fragment of said DNA molecule may or may not overlap with a targeting sequence. Said DNA molecule functions as a transformation vector.

The invention provides a number of transformation vectors and methods for their application. According to this invention, incomplete gene expression cassettes, “gene fragments”, allow for the generation of a selective advantage as well as for the expression of desired foreign sequences. The invention also includes the use of novel selection inhibitors for plastid transformation and, most importantly, the possibility of performing several successive rounds of transformation enabling the insertion of an unlimited number of transgenes into one or more loci of the plastome without accumulation of resistance genes.

The processes and vectors of this invention can be used to target a transcribed region, a sequence leading to a transcript or a sequence not leading to a transcript, like a promoter. They are particularly well suited to target transcribed regions of a plastome without undue interference with plastid gene function.

Said sequence element of the host plastid which is required for operation of said gene fragment in a transformed plastid may be any sequence of the host plastid. It may be a transcribed sequence or non-transcribed sequence. Preferably, it is a control sequence necessary for gene expression.

Gene fragments miss wholly or partly at least one or more of the following four elements which are comprised by a complete gene expression cassette: (1) a promoter sequence, (2) a 5′ untranslated region (5′-UTR), (3) a coding region which encodes the complete sequence of a protein or an RNA, and (4) a 3′ untranslated region (3′-UTR). Preferably, gene fragments miss at least a promoter sequence, wholly or partly. Preferred embodimens are as follows:

Embodiment 1

Vectors for Gene Fragments Missing Promoter Regions

The first set of vectors for this embodiment of the invention is designed to introduce new DNA sequences into existing operons without disturbing the expression of the original genes. This may be achieved by using short homologous spacer regions with and/or without processing signals as well as synthetic spacer regions designed according to effective ribosomal binding sites (Shinozaki & Sugiura, 1982) or viral translation initiation sites shown to be active in plastids (Hefferon et al., 2000). The constructs may contain uidA as an expression marker and aadA as a selection marker. Both may be preceded by different spacer regions. The expression as well as the selection marker can easily be replaced by other sequences of interest or expanded with additional sequences.

The second set of vectors is designed to insert new sequences behind the 3′-UTR of strongly expressed and inefficiently terminated monocistronic genes (Stem & Gruissem, 1987). Such a process may generate new multicistronic operons wherein the strong promoter of the original monocistronic gene initiates transcription. As in the first set of vectors, spacer regions may precede the expression and selection markers. Optionally, this new operon may be succeeded by an efficient 3′-UTR.

Embodiment 2

Vectors for Gene Fragments Missing Complete Coding Regions and Promoter Regions

This second embodiment of the invention may be utilised for the creation of plastome point mutations, which confer resistance to various inhibitors or antibiotics. These inhibitors, which can be used for selection in plastid transformation, include spectinomycin and streptomycin (Maliga et al. 1973, Svab et al. 1990), lincomycin (Cséplö and Maliga 1982, Cséplö et al. 1988), atrazine (Sato et al. 1988), tentoxin (Durbin and Uchytil 1977, Klotz 1988), metribuzin (Schwenger-Erger et al. 1993, Perewoska et al. 1994, Schwenger-Erger et al. 1999) and diuron (Renger 1976, Wolber et al. 1986, Sato et al. 1988, Erickson et al. 1989) as well as functional analogs or derivatives thereof. Point mutations can be introduced into the plastome by homologous recombination (Svab et al. 1990). Vectors may contain mutant sequences in one of the flanks required for homologous recombination. Such flanks may start in that part of the coding region, which contains the point mutation(s), i.e. the vectors neither contain a promoter, nor a 5′-UTR, and contain only part of the coding region of the mutant gene in question. One or more desired foreign sequences may then be included without a separate promoter as described for embodiment 1, and vectors may be completed by the addition of a second flank for homologous recombination.

Embodiment 3

Vectors for Gene Fragments (Missing Complete Coding Regions and Promoter Regions), which Allow the Insertion of One or More Transgene(s) into Plastome Loci Different from the Sequence Used for Selection

This third embodiment of the invention may utilise plastome point mutations which confer resistance to various inhibitors as described for embodiment 2. A gene fragment carrying the respective point mutation(s) provides the homologous region necessary for recombination. The sequence(s) of interest may be located in a different position on the same vector without being operably linked to the marker fragment. These sequence(s) of interest, which may carry flanks for homologous recombination into any desired locus of the plastome, may be expressed by a) insertion of the new sequences into existing operons without disturbing the expression of the original genes or b) by insertion of the new sequences behind the 3′-UTR of strongly expressed and inefficiently terminated monocistronic genes (Stern & Gruissem, 1987) or c) by modifying a pre-existing cistron, as described for embodiment 1. The sequence(s) of interest and the marker fragment carrying the point mutation(s) may also be located on two physically separated plasmid vectors. The gene fragment with the point mutation(s) and the DNA fragment with the sequence(s) of interest may be integrated into the plastome by two independent recombination events (co-transformation). Co-transformation was reported to be efficient in plastid transformation (Carrer and Maliga 1995). In comparable experiments it was found that the aadA and GFP genes can be co-transformed from separate plasmids with an efficiency of 30%.

Embodiment 4

Vectors for Gene Fragments Modifying, Removing or Replacing Regulatory Elements

In this fourth embodiment of the invention sequence elements may be removed from or introduced into the plastome in order to modify the regulation of expression of sequences contained in the plastome. Such sequence elements may represent complete 5′- or 3′-regulatory sequences, parts of them, or new elements modifying gene expression. The introduced sequences may for instance be promoters, ribosomal binding sites, sequences modifying mRNA stability, or viral translation initiation sites shown to be active in plastids (Hefferon et al., 2000). The function of the sequences may also be to prevent the expression of targeted sequences by separating them from their regulatory elements. This method can also be used for modification of expression of sequences introduced in previous transformations.

Embodiment 5

Gene Fragments for Changing a Pre-Existing Cistron Without Creating an Additional Cistron

In an important embodiment, said DNA modification is designed for changing a pre-existing cistron and said fragment of a gene of interest is preferably a fragment of a heterologous gene. In this embodiment, said DNA change comprises insertion of said fragment of a gene of interest in a pre-existing cistron, whereby no additional cistron (see Definitions) is created. The fragment of a gene of interest may be inserted anywhere in a pre-existing cistron. The fragment of a gene of interest may also be joined with a pre-existing cistron at the beginning or, preferably, at the end of a pre-existing cistron. Said changed cistron is preferably designed for forming a hybrid messenger RNA comprising RNA sequence derived from host plastid DNA and RNA sequence derived from DNA of said fragment of a gene of interest. Said hybrid messenger RNA may encode one or multiple heterologous polypeptides or proteins. Translation of all or a part of said hybrid messenger RNA may lead to a fusion protein. Said fusion protein may comprise a gene product of the preexisting cistron and a gene product of the fragment of the gene of interest (cf. examples 12 and 13). Said fusion protein may comprise multiple heterologous polypeptide sequences. The fragment of a gene of interest or said DNA molecule may have one or more sequences each encoding a proteolytic cleavage site allowing cleavage of a desired protein out of the fusion protein after expression. Said proteolytic cleavage sites may be autocatalytic. As an example, the fragment of a gene of interest may be provided with intein sequences to allow post-translational excision of a protein of interest out of the expressed fusion protein (cf. example 12).

The fragment of a gene of interest may also be inserted in a pre-existing cistron that codes for RNA that is not translated like ribosomal RNA (rRNA). In this case, transcription will produce a hybrid mRNA comprising the transcription product of the preexisting cistron and the mRNA of the fragment of a gene of interest. The fragment of a gene of interest is preferably provided with sequences enabling translation like a ribosome binding site in order to allow production of a protein of interest encoded by said fragment of a gene of interest. The fragment of a gene of interest may e.g. be inserted in any pre-existing cistron encoding any rRNA. Preferably, said insertion in said pre-existing cistron encoding an rRNA does not destroy a vital function of the plastid. A preferred example of such a cistron is sprA (Sugita et al., 1997; Vera and Sugiura, 1994).

Embodiment 6

Generation of a Functional Gene of Interest Using Multiple Transfomation Steps

In this embodiment, stably transformed plastids of functional cells or multicellular plants are obtained in a first process or step of transformation according to a process of the invention. In this first step, the plastome is endowed with a first gene fragment that is non-functional, i.e. does preferably not produce a trait. Examples for such a non-functional gene fragment are: a part of a coding region, a sequence that serves as a targeting sequence (“landing pad”) for homologous recombination of the second transformation step etc. Selection is performed after the first transformation step to obtain functional cells with stably transformed plastids. Also, multicellular plants may be regenerated. In a second step, cells or plants having stably transfomed plastids are transformed with a second DNA molecule via homologous recombination for enabling a second DNA modification, whereby said second DNA molecule comprises a gene of interest or, preferably, a fragment thereof requiring for expression in a transformed plastid a sequence element of the host plastid or a sequence element of a fragment of a gene introduced in said first transformation step. When necessary, said second transfomation step may be followed by a selection procedure as defined in steps (b) and (c) of claim 1. In this embodiment, said DNA modifications of said first and said second step jointly generate an operon or a cistron with a desired function (cf. example 14). Said desired function may e.g. be expression of a desired gene of interest that cannot be expressed if one of said two transformation steps is omitted. Said second DNA molecule may additionally contain a further gene of interest like a reporter gene. One important advantage of this embodiment is that it greatly contributes to biological safety, since a single transformation process does not generate a functional gene of interest.

The principle of the above-described two step process may easily be extended to more than two transformation steps (e.g. three steps) that jointly generate a desired function.

The various embodiments of this invention may also be performed simultaneously (e.g. by co-transformation) or consecutively and together represent highly versatile tools for plastid genome modification unthinkable before. They allow plastid modification nearly at will. An example is the insertion of a whole biosynthetic pathway in a plastid.

Definitions

The following definitions are given in order to clarify the meaning of certain terms used in the description of the present invention.

3′-UTR: transcribed but not translated region of a (→) gene, downstream of a (→) coding region; in (→) plastid (→) genes, the 3′-UTR inter alia serves to stabilise the mRNA against 3′ to 5′ exonucleolytic degradation;

5′-UTR: transcribed but not translated region of a (→) gene, upstream of a (→) coding region; in (→) plastid (→) genes, the 5′-UTR contains sequence information for translation initiation (ribosome binding site, (→) RBS) close to its 3′ end;

aadA: (→) coding region of bacterial aminoglycoside adenyl transferase, a frequently used protein, that detoxifies antibiotic (→) selection inhibitors spectinomycin and/or streptomycin;

chloroplast: (→) plastid containing chlorophyll;

cistron: a DNA sequence at a locus encoding one functional RNA (as opposed to messenger RNA) or one polypeptide; the polypeptide encoding sequence may contain one or more non-coding regions ((→) introns). More than one cistron can be organized in an (→) operon.

coding region: nucleotide sequence containing the information for a) the amino acid sequence of a polypeptide or b) the nucleotides of a functional RNA; coding regions are optionally interrupted by one or more (→) intron(s);

desired gene (sequence): modified or newly introduced sequence: the purpose of a (→) transformation attempt;

flank, flanking region: DNA sequences at the 5′ and 3′ ends of inserts in a (→) plastid (→) transformation (→) vector, which mediate integration into the target (→) plastome of sequences between the flanks by double reciprocal (→) homologous recombination. By the same mechanism, sequences can be modified or removed from the target (→) plastome. Thus, the flanks of the (→) plastid (→) transformation (→) vector determine, where changes in the target (→) plastome are generated by (→) transformation;

gene expression: process turning sequence information into function; in ( ) genes encoding messenger RNA, which is subsequently translated into a polypeptide; in (→) genes encoding RNA, the (→) promoter-mediated activity of RNA polymerase generates the encoded RNA;

gene fragment: nucleotide sequence(s) encoding less elements, than are required to secure function independently, i.e. autonomous function like expression;

(→) genes are organised in (→) operons, which contain at least one complete (→) coding region;

in (→) genes encoding polypeptides, these elements are: (1) a (→) promoter, (2) a 5′ untranslated region ((→) 5′-UTR), (3) a (→) complete coding region, (4) a 3′ untranslated region ((→) 3′-UTR);

in (→) genes encoding RNA, the (→) 5′-UTR and the (→) 3′-UTR are missing;

in (→) operons consisting of more than one (→) coding region, two subsequent complete (→) coding regions are separated by a (→) spacer; (→) promoter, (→) 5′-UTR, and (→) 3′-UTR elements are shared by the (→) coding regions of that operon;

gene(s): nucleotide sequence(s) encoding all elements, which are required to secure function e.g. expression independently;

genes are organised in (→) operons, which contain at least one complete (→) coding region;

in (→) genes encoding polypeptides, these elements are: (1) a (→) promoter, (2) a 5′ untranslated region ((→) 5′-UTR), (3) a complete (→) coding region, (4) a 3′ untranslated region ((→) 3′-UTR);

in (→) genes encoding RNA, the (→) 5′-UTR and the (→) 3′-UTR are missing;

in (→) operons consisting of more than one (→) coding region, two subsequent complete (→) coding regions are separated by a (→) spacer, and (→) promoter, (→) 5′-UTR, and (→) 3′-UTR elements are shared by the (→) coding regions of that (→) operon;

genome: Complete DNA sequence of a cell's nucleus or a cell organelle;

homologous recombination: process leading to exchange, insertion or deletion of sequences due to the presence of (→) flanks with sufficient sequence homology to a target site in a (→) genome;

insertion site: locus in the (→) plastome, into which novel sequences are introduced;

intergenic region: sequences between two (→) genes in a (→) genome; such region occur as (→) interoperonic regions or as (→) intraoperonic regions, in which case they are also called (→) spacers;

interoperonic region: sequences between (→) operons;

intraoperonic region: sequences inside (→) operons;

intragenic region: sequences inside a (→) gene;

intron: sequence interrupting a (→) coding region;

non-transcribed region: a genome region, which can only be targeted for functional expression by a vector with an autonomous sequence including all control sequences required for expression;

operon: organisational structure of several (→) genes sharing a promoter;

plant(s): organism(s) that contain(s) (→) plastids in its (their) cells; this invention particularly relates to multicellular (→) plants; these include the group of gymnosperms (such as pine, spruce and fir etc.) and angiosperms (such as the monocotyledonous crops maize, wheat, barley, rice, rye, Triticale, sorghum, sugar cane, asparagus, garlic, palm tress etc., and non-crop monocots, and the dicotyledonous crops tobacco, potato, tomato, rape seed, sugar beet, squash, cucumber, melon, pepper, Citrus species, egg plant, grapes, sunflower, soybean, alfalfa, cotton etc.), and no-crop dicots as well as ferns, liverworths, mosses, and multicellular green, red and brown algae;

plastid(s): organelle(s) with their own genetic machinery in (→) plant cells, occurring in various functionally and morphologically different forms, e.g. amyloplasts, (→) chloroplasts, chromoplasts, etioplasts, gerontoplasts, leukoplasts, proplastids etc;

plastome: complete DNA sequence of the (→) plastid;

processing signal: RNA transcripts often require maturation (“processing”, i.e. excision/religation events) before reaching functionality; the information for processing lies within “processing signals” in the DNA sequence;

promoter: nucleotide sequence functional in initiating and regulating transcription;

RBS, ribosomal binding site: DNA sequence element upstream of the (→) translation start codon of a (→) coding region, that mediates ribosome binding and translation initiation from the respective RNA transcript; RBS elements are either part of (→) 5′-UTRs or of (→) spacers;

selection inhibitor: chemical compound, that reduces growth and development of non-transformed cells or organelles stronger than that of transformed ones;

spacer: (→) intergenic region between the 3′ end of one (→) coding region and the 5′ end of another (→) coding region of an (→) operon;

termination: in the description of this invention, “termination” relates to discontinuation of transcription of RNA from a DNA sequence;

transcribed region: a genome region, which can be targeted for functional expression by a vector with a non-autonomous sequence, that relies at least partly on a sequence of the transcribed region. Within such a transcribed region the position targeted may be an intragenic position or an intraoperonic spacer position or a position directly downstream of an operon;

transformation vector: cloned DNA molecule that was generated to mediate (→) transformation of a (→) genome;

transformation: process leading to the introduction, the excision or the modification of DNA sequences by treatment of (→) plants or plant cells including the use of at least one (→) transformation vector;

transgene: DNA sequence derived from one (→) genome, introduced into another one;

translation start codon: sequence element, that encodes the first amino acid of a polypeptide;

translation stop codon: sequence element that causes discontinuation of translation;

uidA: (→) coding region of bacterial β glucuronidase, a frequently used reporter protein;

### SHORT DESCRIPTION OF THE FIGURES

FIG. 1 is a schematic view of vector pIC500.

FIG. 2 is a schematic view of vector pIC569.

FIG. 3 is a schematic view of vector pIC574.

FIG. 4 is a schematic view of vector pIC579.

FIG. 5 is a schematic view of vector pIC576.

FIG. 6 is a schematic view of vector pIC584.

FIG. 7 is a schematic view of vector pIC583.

FIG. 8 is a schematic view of vector pIC582.

FIG. 9 is a schematic view of vector pIC570.

FIG. 10 is a schematic view of vector pIC581.

FIG. 11 is a schematic view of plastid transformation vector pIC588 and its target site in tobacco plastid DNA.

FIG. 12 is a schematic view of plastid transformation vector pIC587 and its target site in tobacco plastid DNA.

FIG. 13 is a schematic representation of alternating transformations with vectors conferring spectinomycin or streptomycin resistance.

FIG. 14 is a schematic view of plastid transformation vector pIC591 and its target site in tobacco plastid DNA.

FIG. 15 illustrates a transformation vector for achievement of atrazine resistant plants.

FIG. 16 illustrates a transformation vector for achievement of tentoxin resistant plants.

FIG. 17 is a schematic representation of alternating transformations with aadA and aph6 based on inactivation of the selection marker by exchange of regulatory elements.

FIG. 18 is a schematic view of vector pICINT (cf. example 12).

FIG. 19 is a schematic view of vector pICFUS (cf. example 13).

FIG. 20 is a schematic view of plastid transformation vector pIC-aphA6-rpl32.

FIG. 21 is a schematic view of plastid transformation vector pIC-uidA-aphA6.

FIG. 22 is a schematic view of plastid transformation vector pIC-uidA-aphA6-frag.

### DETAILED DESCRIPTION OF THE INVENTION

Plastids have their Own Genetic Machinery

According to generally accepted knowledge, two classes of cell organelles, i.e. plastids, and mitochondria, are derived from initially independent prokaryotes that were taken up into a predecessor of present day eukaryotic cells by separate endosymbiotic events (Gray, 1991). As a consequence, these organelles contain their own DNA, DNA transcripts in the form of messenger RNA, ribosomes, and at least some of the necessary tRNAs that are required for decoding of genetic information (Marechal-Drouard et al., 1991).

While, shortly after endosymbiotic uptake, these organelles were genetically autonomous, since they contained all the elements necessary to drive prokaryotic life, this autonomy was reduced during evolution by transfer of genetic information to the cell's nucleus. Nevertheless, their genetic information is of sufficient complexity to make such cell organelles an attractive target for gene technology. This is particularly the case with plastids, because these organelles still encode about 50% of the proteins required for their main function inside the plant cell, photosynthesis. Plastids also encode their ribosomal RNAs, the majority of their tRNAs and ribosomal proteins. In total, the number of genes in the plastome is on the order of 120 (Palmer, 1991). The vast majority of proteins that are found in plastids are, however, imported from the nuclear/cytosolic genetic compartment.

Plastids can be Genetically Transformed

With the development of general molecular cloning technologies, it became soon possible to genetically modify higher plants by transformation. The main emphasis in plant transformation was and still is on nuclear transformation, since the majority of genes, ca. 26.000 in the case of Arabidopsis thaliana, the complete sequence of which was recently published (The Arabidopsis Genome Initiative, 2000), is found in the cells nucleus. Nuclear transformation is easier to achieve, since biological vectors such as Agrobacterium tumefaciens were available, which) could be modified to efficiently enable nuclear transformation (Gelvin, 1998). In addition, the nucleus is more directly accessible to foreign nucleic acids, while the organelles are surrounded by two envelope membranes that are, generally speaking, not permeable to macromolecules such as DNA.

A capability of transforming plastids is highly desirable since it makes use of the enormous gene dosage in these organelles—more than 10000 copies of the plastome may be present per cell—that bears the potential of extremely high expression levels of trangenes. In addition, plastid transformation is attractive because plastid-encoded traits are not pollen transmissible; hence, potential risks of inadvertent transgene escape to wild relatives of transgenic plants are largely reduced. Other potential advantages of plastid transformation include the feasibility of simultaneous expression of multiple genes as a polycistronic unit and the elimination of positional effects and gene silencing that may result following nuclear transformation.

Methods for transformation of plastids have been developed, initially for a single cell green alga (Boynton et al., 1988), and subsequently also for higher plants. To date, two different methods are available, i.e. particle bombardment of tissues, in particular leaf tissues (Svab et al., 1990), and treatment of protoplasts with polyethylene glycol (PEG) in the presence of suitable transformation vectors (Koop et al., 1993). Both methods mediate the transfer of plasmid DNA across the two envelope membranes into the organelle's stroma.

Plastid Transformation Vectors Share a Common Structure

Besides the development of methods to introduce DNA into the organelle, two further requirements had to be met for achievement of plastid transformation, namely the use of plastid sequence elements regulating gene expression, and enabling site directed integration of novel or modified sequences by homologous recombination. Homologous recombination requires the presence in transformation vectors of flanks that are derived from plastome sequences upstream and downstream of the targeted insertion site (Zoubenko et al., 1994). As a consequence of these requirements, plastid transformation vectors used for the introduction of foreign genes into the plastome of higher plant plastids share the following elements: (1) a 5′ flank, (2) a promoter sequence, (3) a. 5′ untranslated region, (4) a coding region, which encodes the complete sequence of a protein gene or a non protein gene (such as an RNA gene), and (5) a 3′ untranslated region, and (6) a 3′ flank. In addition (7), a plastid origin of replication was postulated to be required (U.S. Pat. No. 5,693,507).

Surplus Stretches of Homology Lead to Genetic Instability

The above sequences (1) through (3), and (5) through (7) are of plastid origin and are here contemplated as potentially serving as substrates for homologous recombination, which is known to be very efficient in plastids (Kavanagh et al., 1999). Homologous sequences in addition to those that are required for integration may cause genetic instability, particularly as long as transformed and untransformed plastomes coexist inside the same organelle (Eibl et al., 1999).

The Novel Vectors of this Invention Avoid Surplus Homologous Sequences

The present invention can be characterised as having two key features: (1) novel plastid transformation vectors are disclosed, in which sequences or genes of interest are included that comprise gene fragments rather than complete expression cassettes, and (2) a number of novel selection schemes are described.

The novel vectors consist of fewer elements than conventional plastid transformation vectors. In different embodiments, at least one of the following elements are missing wholly or partly: promoter(s), 5′ untranslated region, complete coding region, and 3′ untranslated region. The novel vectors do not require homologous flanks that are separate from a coding region of a gene conferring a selective advantage. In other words, a homologous flank used as a targeting sequence may overlap at least partly with a sequence of one of the above elements. Embodiments are also provided, wherein only one of the above elements is included in the transformation vector. Thus, as an example, replacement by homologous recombination of just a partial coding region can confer resistance to a selective agent. Likewise, replacement, deletion or modification of just a single regulatory element such as a promoter, a translation start signal or a UTR may lead to changed gene expression patterns.

As the consequence, due to the usage of less homologous sequences, this invention makes possible stable plastid modifications, particularly, stable insertions of transgenic sequences such that they can be functionally inherited and can be incorporated in stable cell lines and plants.

Plastid Genome Structure Makes the Use of Gene Fragments Possible

The feasibility of these approaches is based on at least three features of the plastid genome and its expression. (1) Homologous recombination is extremely efficient (Kavanagh et al., 1999) and requires only very limited lengths of homologous flanks (Staub & Maliga, 1994). Therefore, flanks can be used that do not consist of complete expression cassettes. (2) The majority of plastid genes are organised in operons containing more than one cistron (Shinozaki et al., 1986). Therefore, additional cistrons can be introduced into existing operons. (3) ‘Read through’ transcription is common for plastid genes (Stern & Gruissem, 1987). Therefore additional cistrons can be introduced behind existing operons.

Vectors of this Invention are Easier to Generate and Improve Genetic Stability

Using gene fragments rather than complete expression cassettes comprising multiple sequence elements considerably simplifies vector construction work. Every element needs to be cloned and, as a rule, supplied with suitable restriction sites separately. Such a procedure is time consuming since it requires multiple cloning steps. Gene fragment vectors can be generated in considerably less time, since elements that are already present in the plastome are employed to mediate expression of novel sequences.

Even more importantly, since gene fragment vectors contain fewer regions of homology than conventional plastid transformation vectors, they improve genetic stability. Undesired loss of sequences (Eibl et al., 1999) is avoided.

Vectors of this Invention Provide Novel Selection Schemes

The aadA gene is the only selectable marker gene that is used routinely (Heifetz, 2000), and the nptII gene conferring resistance to kanamycin is the only alternative that has been shown to function in higher plant plastid transformation (Carrer et al., 1993). Since neither the aadA nor the nptII gene can be used universally, the number of higher plant species, that have been transformed in the plastome is still very low (Heifetz, 2000). Plastid transformation in higher plants cannot at present be exploited to its full potential. Procedures like subsequent or simultaneous introduction of more than one desired sequence require more selectable marker genes. The vectors of this invention overcome the shortage of selectable marker genes. Novel selection inhibitors for plastid transformation that are described here include lincomycin, tentoxin, atrazin, metribuzin, and diuron. It is noteworthy that among the inhibitors of this list, the last four agents are not antimicrobial antibiotics. Use of antimicrobial antibiotics that are of therapeutical significance for treatment of diseases in humans or animals is subject of critical public debate and generates problems with general acceptance of plant gene technology (Daniell, 1999).

Vectors of this Invention Provide the Option to Reuse Selection Markers by Alternating Between Various Markers

A further application of gene fragments for plastid transformation according to this invention is the possibility to alternate between two selection inhibitors, for which point mutations conferring the respective resistances are located within the same gene. The mutations may be located at two different positions within one gene or at the same position. As an example, the vector used for the first transformation may contain a gene fragment which contains only one point mutation. The vector used for the second transformation contains a gene fragment which may include the positions of both point mutations, but contains only the second mutation, while it is wild type with respect to the position of the first resistance. By transformation with this second vector the first mutation is removed, while the second one is simultaneously introduced. In a subsequent transformation, the second mutation can be removed again in the same way reintroducing the first mutation. This strategy allows multiple subsequent transformations of one plastome without accumulation of multiple resistances in the plant; only a single resistance will be present after each step. The method can be applied to any gene, for which mutations conferring resistance to at least two different inhibitors are found. Examples described here include the rm16 gene (spectinomycin and streptomycin), and the psbA gene (atrazine and metribuzine).

The Vectors of this Invention Allow Introduction of Selection Markers and Sequences of Interest at Independent Loci

If a point mutation or a version of a plastid gene that is modified otherwise is used as a selection marker for plastid transformation, the position of the selection marker in the plastome is fixed. Conventional transformation mediates insertion of further sequences of interest into the same locus as the selection marker, since it uses the same recombination event. The approach of embodiments 3 and 4 (see summary of the invention) allows the separation of the insertion sites of the marker gene and the sequences of interest by using a transformation vector wherein these two features are physically separated. The principle of separation of selection marker and gene of interest has been demonstrated by co-transformation with two independent plasmids (Carrer and Maliga, 1995) and was confirmed by our own experiments using the aadA selection marker gene and the GFP gene on two different plasmids. The efficiency of co-transformation of these two genes was more than thirty percent (Muhlbauer et al., unpublished results).

Our approach is novel, since it combines two independent events of homologous recombination in a single vector. This leads to a higher efficiency, as upon uptake of only one vector molecule into a plastid the presence of both sequences is ensured.

The described method makes different integration sites accessible, which allows, for instance, a different regulation of expression of the sequence(s) of interest e.g. the selection marker gene and/or the gene of interest. In addition, directed mutagenesis of plastid genes can be performed without integration of a marker gene in its immediate vicinity which might disturb the expression of the plastid gene. The method can also be used for simultaneous insertion of a gene of interest at the locus of the selection marker and a second gene of interest at a different locus. Any of the selection markers described in this application can be used for this system.

In combination with alternation between two different selective agents (see above), multiple additional sequences of interest can be inserted at any locus of the plastome, with only one selection marker present in the transformed plant.

The Process of the Invention Allows Changing a Pre-Existing Cistron Without Creating an Additional Cistron by Said Change

Yet another process contemplated in this invention is a process allowing the use of gene fragments for targeted modification of existing cistrons, i.e. a process that does not create an additional cistron. Such a transformation-mediated modification of plastid DNA allows one skilled in the art to create recombinants that, upon transcription, produce a hybrid messenger RNA (mRNA), comprising transcribable parts of the resident gene and of a gene of interest, or a mRNA that uses transcription signals of the resident gene to express mRNA of the gene of interest, abandoning the trascribed part of the resident gene. Such mRNA then may be translated, and the translation of all or part of said mRNA can produce a functional polypeptide. Here, different possibilities are contemplated. If such a hybrid mRNA involves a plastid gene that per se is not translated (for example, a ribosomal RNA gene), the hybrid RNA may still be translated and yield a protein product from the part of the chimera that encodes a translatable sequence. The non-translatable part of such a hybrid RNA may also still preserve its function. If, on the other hand, the resident gene involved in recombination is translatable, translation of the hybrid RNA will yield a fusion protein. Depending on the functionality of the fusion protein, it may be able to both perform the function of the resident gene in such a modified form as well as provide a new added function encoded in the fragment of the gene of interest. If the fusion product affects negatively the function of the plastid gene, it is conceivable to design the process by targeting a resident gene whose function is despensable (e.g. sprA). Alternatively, a fusion protein may be engineered that contains a cleavage sequence between the two parts of the fusion protein, which cleavable site can then be treated with a protease or other factors that allow to separate the two components of the fusion protein subsequent to protein translation.

Among the examples for such a fusion protein approach as provided herein is the following one: the coding portion of a reporter gene is fused to the ycf9 gene to generate one large chimeric peptide. Selection may be achieved by co-transformation with a gene fragment having a point mutation (Spec/Strep). In this simpler version, the fusion protein generates a new trait because of the expression of the coding part of the gene fragment of interest. The function of the protein encoded by the ycf9 gene, on the other hand, is dispensable. A more sophisticated version of this an approach is exemplified by the following specific embodiment: a modified intein is introduced into the psbA gene which is highly expressed or in another suitable gene. The endonuclease part of the intein may be removed and exchanged for the aadA selectable marker. Intein-mediated splicing will release the AADA-Protein.

This aspect of our invention has a number of potential uses. One most obvious advantage of the process designed to create translational fusions instead of additional transcriptional or translational units is that the resultant plastid chromosome does not have additional regulatory sequences and additional streches of homology, thus the stability and functioning of such plastome is much less affected as a result of manipulations. One is also able to use any strong promoter and any transcriptionally active area of the chromosome for dual or multiple purposes. One is further able to replace some plastid genes whose function is not essential or whose function can be moved under a nuclear control. Use of the transcription elements with specific expression profiles in their natural chromosomal environment allows to link expression of the gene of interest with a specific physiological or developmental conditions of the organism, for example light, and thus have a controlled expression.

Process of Using Two or More Gene Fragments to Generate a Functional Gene of Interest in Multiple Steps

Yet another process contemplated in this invention is a process allowing the use of two or more of fragments of a gene of interest, which fragments, being non-functional when separated, are restored into a functional gene upon a process that involves two or more independent DNA integration steps. Such a process would require at least two separate integration events, with the end result being a correctly assembled transcriptionally and translationally functional unit that may or may not require some parts of the resident plastid DNA for its functioning.

In an experiment that exemplifies the above mentioned invention, a fragment of a marker gene (aphA-6) is inserted, using co-transformation e.g. with the Spec point mutation, in the first transformation step. In a second transformation step, a reporter gene is inserted and functional aphA6 is restored to a fully functional state. The second-step selection may be achieved by using resistance to Kanamycin, thus demonstrating functionality of the gene of interest.

The advantages and utilities of such a gene reconstruction process based on multiple steps are obvious. Transformation of the wild-type plastome is in many cases difficult to achieve in one transformation step, as one has to design specific homology regions on a vector, depending on which specific plastid chromosome and which specific insertion site is contemplated.

Therefore, a mature plastid transformation technology will be a two- or a multi-step process, that modifies the wild-type chromosome so as to create a more advanced “landing pad” for the subsequent insertions. Such a technology: a) is more easy and versatile (i.e. based on one standard vector for secondary transformations), b) allows to easily inactivate any unwanted gene (i.e. selection marker gene) introduced in the first transformation step, and c) allows to control the process better as only legitimate recombinants will yield a functioning gene of interest, d) allows to eliminate problem of contaminations with nuclear transformants, as the function of gene of interest is restored only upon homologous recombination, i.e. the process requires a specific integration and the presence of another essential gene fragment for the function to be restored.

The teaching of this invention may be used to produce plant cells and plants having stably or non-stably transformed plastids. Plastids of many different plant species may be transformed. The invention is applicable to monocots and dicot plants. Crop plant are particularly preferred. Examples of such crop plants are maize, rice, wheat, oat, rye, barley, soybean, tobacco, tomato, potato, grape, peanut, sweet potato, alfalfa, soghum, pea, and cotton.

### EXAMPLES

The invention will be further described by reference to the following detailed examples. These examples are provided for purposes of illustration only, and are not intended to be limiting. Standard recombinant DNA and molecular cloning techniques used here are well known in the art and are described by Ausubel, 1999, Maniatis et al., 1989 and Silhavy et al., 1984.

### Example 1

### Construction of a Vector for Insertion of Sequences Into an Existing Transcription Unit (psbA) and Plastid Transformation Therewith

Construction of Plastid Transformation Vector pIC500

1 nmol of linker 1P, 1 nmol of linker 2M, 20 nmol dNTP, 50 U Klenow-polymerase are incubated in 100 μl of 1× Y⁺-buffer (MBI, Vilnius, Lithuania) for 30 min. at 34° C. The polymerase is inactivated for 10 min. at 70° C., cooled to 34° C. and the resulting double-stranded oligonucleotide is restricted for 2 h at 34° C. with 50 U Pael (MBI). 50 U EcoRI are added and the Y⁺-buffer concentration is increased to 2× in a total volume of 200 μl. After 1 h incubation at 34° C. the mixture is purified with the PCR-purification-kit from Qiagen (Hilden, Germany) resulting in 30 μl of the purified 117 bp linkerE/P. 1 μg of plasmid pUC19 (MBI) is restricted 2 h at 34° C. with 50 U Pael (MBI) in 100 μl 1× Y⁺-buffer (MBI) and additional 30 min. with 50 U EcoRI (MBI) in 200 μl 2× Y⁺-buffer. The restricted plasmid is treated with 5 μl alkaline phosphatase (Roche, Basel, Switzerland) for 30 min. at 34° C. and finally purified with the PCR-purification-kit from Qiagen resulting in 30 μl of the restricted plasmid pUC-E/P/AP. 1 μl of the linkerE/P and 5 μl of the plasmid pUC-E/P/AP are ligated with 0.5 μl T4-ligase (Promega, Madison, Wis., USA) in 20 μl of 1× T4-ligation-buffer (Promega) at 4° C. over night resulting in cloning vector pIC500 (FIG. 1).

Electrotransformation of pIC500 into E. coli Cells

Preparation of electrocompetent cells: 1 liter of LB-medium (1.% (w/v) casein hydrolysate, 0.5% (w/v) yeast extract, 0.5% (w/v) NaCl) is inoculated 1:100 with fresh overnight culture of E. coli JM109 cells (Promega, Madison, Wis., USA). The cells are grown at 37° C. with shaking at 220 rpm to an optical density of 0.5 at 600 nm. The cells are chilled on ice for 20 min. and centrifuged for 15 min. (4000 rpm, 4° C.). The supernatant is removed and the pellet is resuspended in 1 liter of ice-cold sterile 10% (v/v) glycerol. The cells are centrifuged two times as described before, resuspending the cells in 500 ml and 20 ml of ice-cold sterile 10% (v/v) glycerol, respectively. The cells are centrifuged an additional time and the pellet is resuspended in 2 ml of ice-cold sterile 10% (v/v) glycerol. This suspension is frozen in aliquots of 80 μl and stored at −80° C.

Electrotransformation using the Bio-Rad (Hercules, Calif., USA) Micro Pulser electroporation apparatus: The electrocompetent cells are thawed on ice. 40 μl of the cell suspension are mixed with 2 μl of the ligation mixture and transferred into a prechilled, sterile 0.2 cm cuvette (Bio-Rad). The suspension is shaken to the bottom and the cuvette is placed into the chamber slide. The chamber slide is pushed into the chamber and the cells are pulsed at 2.5 kV. The cuvette is removed from the chamber and the cells are suspended in 1 ml of SOC-medium (2% (w/v) casein hydrolysate, 0.5% (w/v) yeast extract, 10 mM NaCl, 2.5 mM KCl, 10 mM MgCl₂ and 20 mM glucose). The suspension is shaken for 1 h at 37° C. and 100 μl of the suspension is plated on LB plates containing 150 mg/l ampicillin.

Analysis of Plasmid pIC500

Plasmid DNA from 10 transformants are isolated according to standard procedures. 200 ng of plasmid DNA is digested with 5 U NcoI (MBI) in 20 μl 1× Y⁺-buffer (MBI) for 1 h at 34° C. and subsequently analysed on a 0.8% agarose-gel. Only plasmids with inserted linker are digested with NcoI. 4 of the tested plasmids are digested with NcoI. One of them (pIC5001) is further tested by sequence analysis (MWG, Ebersberg, Germany).

Isolation of N. tabacum DNA.

100 mg fresh leaf tissues of tobacco (Nicotiana tabacum; cv. petite havana) were disrupted (2×1 min at 25 Hz) in 200 μl AP1 buffer (DNeasy plant mini kit, QIAGEN)/1 μl reagent DX (foaming inhibition, QIAGEN) using mixer mill MM 300 (Retsch) in a 1.5 ml microcentrifuge tube with one 3 mm tungsten carbide bead. DNA was then purified using the DNeasy plant mini kit.

Construction of Plasmid pIC519 (aadA-HisTag+3′-UTR rpl32)

The E coli aadA-sequence is amplified under standard PCR conditions using 1 U Pfu-polymerase (Promega), 0.1 μM primer oSH4 (TGAATTCCCATGGCTCGTGAAGCGG), 0.1 μM primer oSH5 (TATGGATCCTTGCCAACTACCTTAGTGATCTC) and 30 ng pFaadA II (Koop et al., 1996) as template. The PCR-mixture is denatured at 95° C. for 5 min., followed by 30 cycles for 30 sec. at 95° C., 45 sec. at 55° C. and 3 min. at 72° C. The synthesis is completed by a final incubation for 7 min. at 72° C. The mixture is purified with the PCR purification kit from Qiagen and digested with 30 U NcoI and 30 U BamHI in a total volume of 100 μl 2× Y⁺-buffer (MBI) for 2 h at 37° C. resulting in aadA-N/B. 1 μg of plasmid pIC500 (isolated from a E. coli JM110 strain) is digested with 30 U NcoI and 30 U BcII in a total volume of 100 μl 2× Y⁺-buffer (MBI) for 2 h at 37° C. The digested plasmid is purified with the PCR purification kit from Qiagen and ligated with the purified PCR-product aadA-N/B using T4-ligase (Promega). 2 μl of the ligation mixture are transformed into electrocompetent E. coli cells resulting in plasmid pIC506. The N. tabacum rpl32 3′-UTR is amplified under standard PCR conditions using 1 U Pfu-polymerase (Promega), 0.1 μM primer oSH32 (ACAAGAGCTCATAAGTAATAAAACGTTCGAATAATT), 0.1 μM primer oSH33 (AATTCCTCGAGTAGGTCGATGGGGAAAATG) and 30 ng N. tabacum DNA as template. The PCR-mixture is denatured at 95° C. for 5 min., followed by 30 cycles for 30 sec. at 95° C., 30 sec. at 50° C. and 1 min. at 72° C. The synthesis is completed by a final incubation for 7 min. at 72° C. The mixture is purified with the PCR purification kit from Qiagen and digested with 30 U SacI and 30 U XhoI in a total volume of 100 μl 1× Y⁺-buffer (MBI) for 2 h at 37° C. resulting in Trpl32-S/X. 1 μg of plasmid pIC506 is digested with 30 U SacI and 30 U XhoI in a total volume of 100 μl 1× Y⁺-buffer (MBI) for 2 h at 37° C. The digested plasmid is purified with the PCR purification kit from Qiagen and ligated with the purified PCR-product Trpl32-S/X using T4-ligase (Promega). 2 μl of the ligation mixture are transformed into electrocompetent E. coli cells resulting in plasmid pIC519.

Construction of Plasmid pIC569 (Flanking Regions psbA-3′)

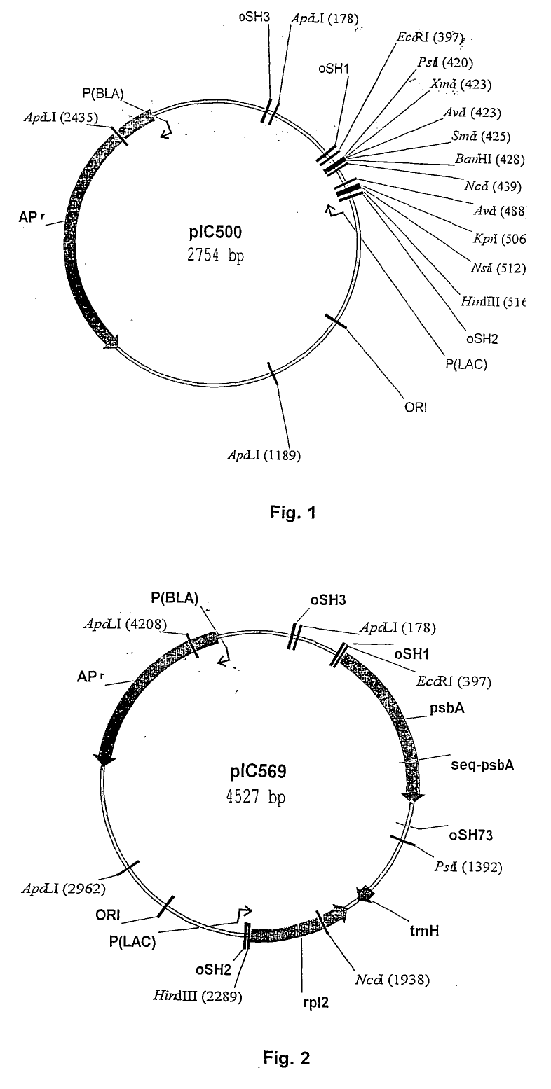

The left flanking region of the N. tabacum psbA-3′-region is amplified under standard PCR conditions using 1 U Pfu-polymerase (Promega), 0.1 μM primer oSH84 (tatagggcccagctataggtttacatttttaccc), 0.1 μM primer oSH85 (catgctgcagcaagaaaataacctctccttc) and 30 ng N. tabacum DNA as template. The PCR-mixture is denatured at 95° C. for 5 min., followed by 35 cycles for 30 sec. at 95° C., 45 sec. at 50° C. and 3 min. at 72° C. Synthesis is completed by a final incubation for 7 min. at 72° C. The right flanking region of the N. tabacum psbA-3′-region is amplified under standard PCR conditions using 1 U Pfu-polymerase (Promega), 0.1 μM primer oSH86 (tttcctgcagftattcatgattgagtattc), 0.1 μM primer oSH87 (ccagaaagaagtatgctttgg) and 30 ng N. tabacum DNA as template. The PCR-mixture is denatured at 95° C. for 5 min., followed by 35 cycles for 30 sec. at 95° C., 45 sec. at 50° C. and 3 min. at 72° C. Synthesis is completed by a final incubation for 7 min. at 72° C. The PCR-products are purified with the PCR-purification-kit from Qiagen resulting in 50 μl LFpsbA and 50 μl RFpsbA respectively. LFpsbA is digested for 2.5 h at 37° C. with 40 U PstI and 40 U Bsp120I in a total volume of 100 μl of 1× Y-buffer (MBI). RFpsbA is digested for 2.5 h at 37° C. with 40 U PstI and 40 U HindIII in a total volume of 100 μl of 1× R⁺-buffer (MBI). Digested LFpsbA and RFpsbA are purified with the PCR-purification-kit from Qiagen resulting in 30 μl LFpsbA-P/B and 30 μl RFpsbA-P/H respectively. 10 μg of plasmid pIC500 are digested with 100 U HindIII, 100 U Bsp120I and 100 U XbaI in a total volume of 300 μl of 1× Y⁺-buffer (MBI) at 37° C. for 3 h. 5 μl alkaline phosphatase (MBI) are added and incubated for 30 min. at 37° C. The digested plasmid is purified on a 0.8% agarose gel. The vector fragment at 2640 bp is excised and purified with the Qiagen gel purification kit resulting in 50 μl pIC500-B/H/AP. 76 fmol pIC500-B/H/AP, 190 fmol LFpsbA-P/B and 170 fmol RFpsbA-P/H are ligated with 0.5 U T4-ligase (Promega) in 10 μl T4-ligase-buffer (Promega) at 4° C. over night. 2 μl of the ligation mixture are transformed into electrocompetent E. coli cells (see above). Transformants with plasmids containing the desired insertion are identified by restriction analysis. The structure of the resulting vector pIC569 is given in FIG. 2.

Construction of Plastid Transformation Vector pIC567

1 μg of plasmid pIC500 is digested with 30 U KpnI in a total volume of 1×KpnI-buffer (MBI) for 3 h at 37° C. The digested plasmid is purified with the PCR purification kit from Qiagen and the KpnI-restriction site is removed by treating the plasmid with 1 U Klenow-polymerase in 1×-Klenow-buffer (MBI) including 50 μM dNTPs for 20 min. at 37° C. The resulting molecule is purified with the PCR purification kit from Qiagen and religated with 0.5 U T4-ligase (Promega) in 10 μl T4-ligase-buffer (Promega) at 4° C. over night. 2 μl of the ligation mixture are transformed into electrocompetent E. coli cells. Transformants containing plasmids with the desired modification are identified by restriction analysis.

Construction of Plasmid pIC574 (SpsaA/B+uidA+SRBS+aadA+Trpl32)

Terminology used for this vector and the constructs described below:

| | |
|---|---|
| S | spacer element |
| SpsaA/B | 25 bp non-coding sequence between psaA and psaB |
| SpsbD/C | 50 bp coding sequence of psbC containing a processing |
| | site |
| Srps19/rpl22 | 60 bp non-coding sequence between rps19 and rpl22 |
| RBS | 18 bp synthetic sequence serving as translation initiation |
| | element |
| T | 3′ UTR (Terminator) |
| Trpl32 | 293 bp fragment downstream of rpl32 coding region |

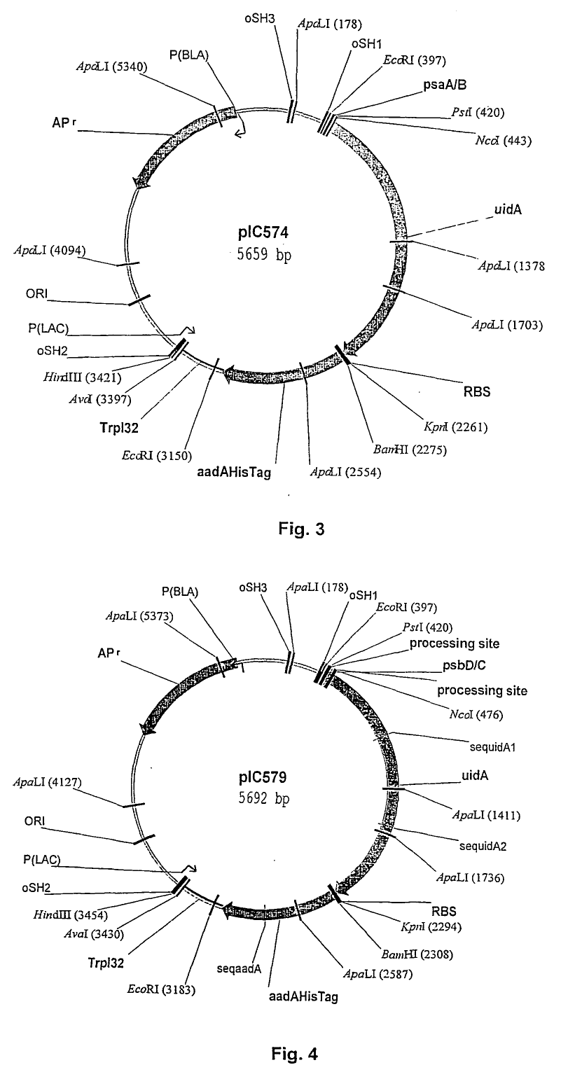

The E. coli aadA-sequence and N. tabacum rpl32-3′-UTR is amplified under standard PCR conditions using 1 U Pfu-polymerase (Promega), 0.1 μM primer oSH88 (ggatccatgcgtgaagcggttatcgccg), 0.1 μM primer oSH33 (aattcctcgagtaggtcgatggggaaaatg) and 30 ng pIC519 as template. The PCR-mixture is denatured at 95° C. for 5 min., followed by 30 cycles for 30 sec. at 95° C., 45 sec. at 55° C. and 3 min. at 72° C. The synthesis is completed by a final incubation for 7 min. at 72° C. The E coli uidA-sequence is amplified under standard PCR conditions using 1 U Pfu-polymerase (Promega), 0.1 μM primer oSH98 (ctgggtaccttattgtttgcctccctgctgcg), 0.1 μM primer oSH74 (catgccatggtccgtcctgtagaa) and 30 ng pRAJ275 (Mike Bevan, gene bank accession U02456.1) as template. The PCR-mixture is denatured at 95° C. for 5 min., followed by 30 cycles for 30 sec. at 95° C., 45 sec. at 50° C. and 3 min. at 72° C. The synthesis is completed by a final incubation for 7 min. at 72° C. The PCR-products are purified with the PCR-purification-kit from Qiagen resulting in 50 μl aadA+Trpl32 and 50 μl uidA respectively. 6 pmol aadA+Trpl32 and 50 pmol oSH97 (ggggtaccagttgtagggagggatccatgcgtgaagc) are incubated with 1 U Taq-polymerase in 1×-Taq-buffer (MBI) including 0.2 mM dNTPs for 20 min. at 72° C. The resulting fragment contains a 5′-RBS-region and is purified with the PCR purification kit from Qiagen resulting in 50 μl RBS+aadA+Trpl32. It is digested with 30 U KpnI and 30 U XhoI in a total volume of 100 μl of 1× Y⁺-buffer (MBI) at 37° C. for 3 h and subsequently purified with the PCR-purification-kit from Qiagen resulting in 50 μl RBS+aadA+Trpl32-K/X. The PCR-product uidA is digested with 30 U KpnI and 30 U NcoI in a total volume of 100 μl of 1× Y-buffer (MBI) at 37° C. for 3 h and subsequently purified with the PCR-purification-kit from Qiagen resulting in 50 μl uidA-N/K. 10 μg of plasmid pIC567 are digested with 100 U PstI and 100 U XbaI in a total volume of 300 μl of 1× Y⁺-buffer (MBI) at 37° C. for 3 h. The digested plasmid is purified on a 0.8% agarose gel. The vector fragment at 2690 bp is excised and purified with the Qiagen gel purification kit resulting in 50 μl pIC567-P/X. 0.6 pmol pIC567-P/X and 300 pmol of the synthetic oligonucleotide SpsaA/B (ctataccatggtgcttttcaaatcctcctagcctgca) are incubated over night at 4° C. with 3 U T4-ligase in 50 μl T4-ligase-buffer (Promega). The mixture is purified with the PCR purification kit from Qiagen and the second strand is subsequently filled in with 1 U Taq-polymerase in 1×-Taq-buffer (MBI) including 0.2 mM dNTPs for 10 min. at 72° C. The resulting plasmid is purified with the PCR purification kit from Qiagen, digested with 30 U NcoI and 30 U XhoI in a total volume of 100 μl of 1× Y⁺-buffer (MBI) at 37° C. for 2 h and subsequently purified with the PCR-purification-kit from Qiagen resulting in 50 μl pIC567-N/X. 25 fmol pIC567-N/X, 25 fmol uidA-N/K and 25 fmol RBS+aadA+Trpl32-K/X are ligated with 0.5 U T4-ligase (Promega) in 10 μl T4-ligase-buffer (Promega) at 4° C. over night. 2 μl of the ligation mixture are transformed into electrocompetent E. coli cells. Transformants with plasmids containing the desired insertion are identified by restriction analysis. The structure of the resulting vector pIC574 is given in FIG. 3.

Construction of Plasmid pIC579 (SpsbD/C+uidA+SRBS+aadA+Trpl32) and pIC576 (Srps19/rpl22+uidA+SRBS+aadA+Trpl32)

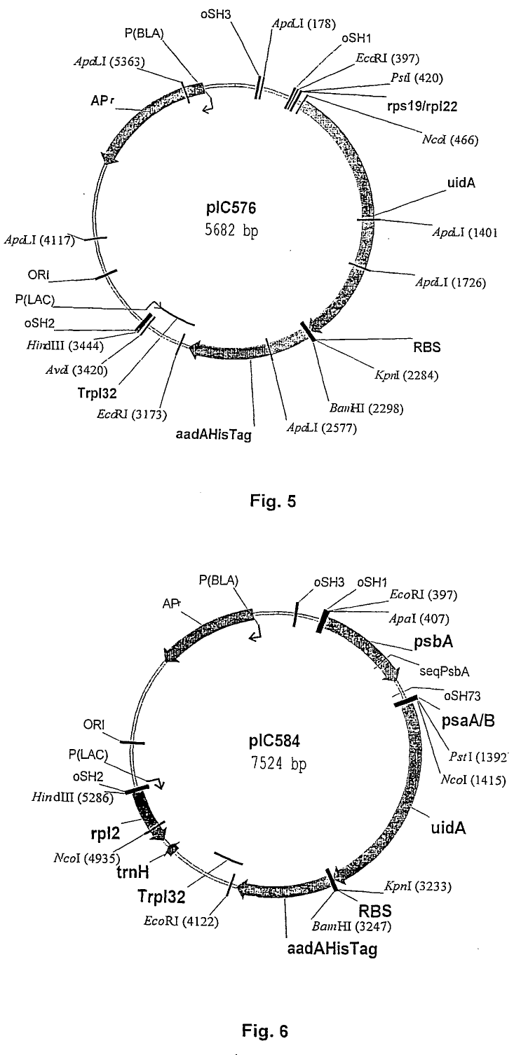

Construction of plasmid pIC579 (FIG. 4) was done as described for pIC574, but using 71 bp of the psbD/C region (ctataccatggggtagaacctcctcagggaatataaggttttgatgaggc tgatcttgagccgccactgca), which contains a processing site, instead of the psaA/B spacer fragment. Accordingly pIC576 (FIG. 5) was constructed using a 60 bp fragment (ctataccatggtttgcctcctactactgaatcataagcatgtagattttttttatctgca) from the noncoding sequence of the rps19/rpl22 intergenic region.

Construction of pIC584, pIC583, and pIC582 (Operon+Flanking Regions)

5 μg pIC574 are digested for 2 h at 37° C. with 30 U PstI and 30 U Mph11031 in a total volume of 100 μl of 1× Y⁺-buffer (MBI). Digested pIC574 is separated on a 0.8% agarose gel. The fragment containing SpsaA/B+uidA+RBS+aadA+Trpl32 is excised and purified with the Qiagen gel extraction kit resulting in 30 μl SpsaAB+uidA+RBS+aadA+Trpl32-P/M. 1 μg pIC569 are digested for 2 h at 37° C. with 30 U PstI in a total volume of 100 μl of 1× O⁺-buffer (MBI). 1 μl of alkaline phosphatase is added to the restriction mixture of pIC569 and incubated for 30 min. Digested pIC569 is purified with the PCR-purification-kit from Qiagen resulting in 30 μl pIC569-P/AP. 75 fmol pIC569-P/AP and 100 fmol SpsaAB+uidA+RBS+aadA+Trpl32-P/M are ligated with 0.5 U T4-ligase (Promega) in 10 μl T4-ligase-buffer (Promega) at 4° C. over night. 2 μl of the ligation mixture are transformed into electrocompetent E. coli cells. Transformants with plasmids containing the desired insertion are identified by restriction analysis. The structure of the resulting vector pIC584 is given in FIG. 6.

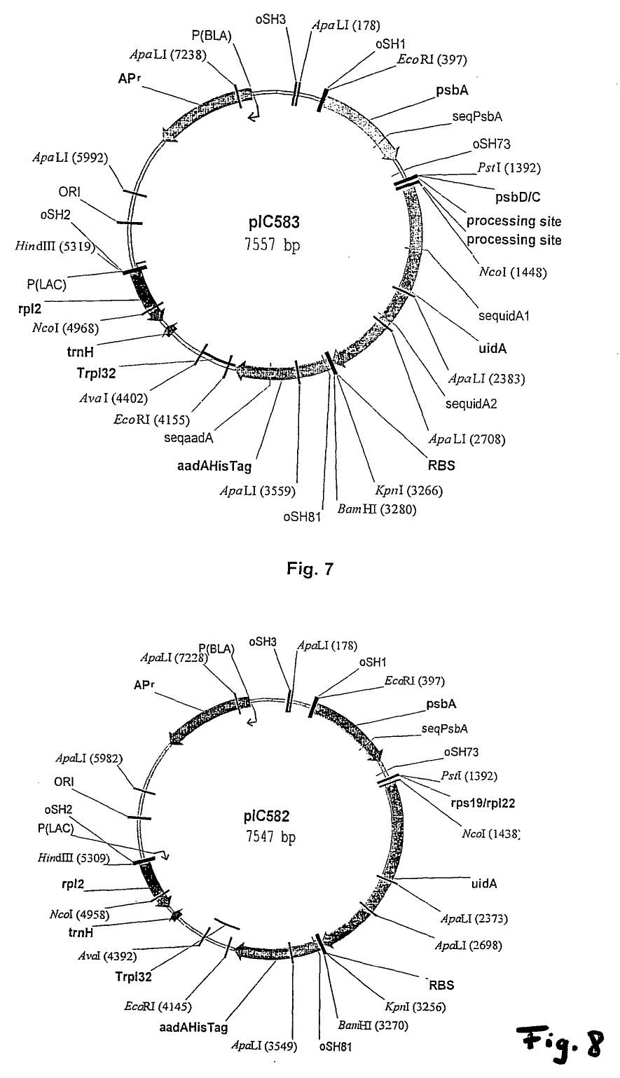

Construction of pIC583 (FIG. 7) and pIC582 (FIG. 8) was done accordingly using Pst I and Mph 1031 digested DNS from pIC579 (for pIC583) and pIC576 (for pIC582) instead.

Transformation of N. tabacum Plastids by Biolistic Delivery

Tobacco seeds (Nicotiana tabacum cv. petite havana) were surface sterilized (1 min in 70% ethanol, 10 min in 5% Dimanin C, Bayer, Leverkusen, Germany), washed 3 times for 10 min in sterile H₂O and put on B5 medium (see below). Plants were grown at 25° C. in a 16 h light/8 h dark cycle (0.5-1 W/m², Osram L85W/25 Universal-White fluorescent lamps).

6 leaves from 4 weeks old, sterile grown Nicotiana tabacum plants are cut and transferred on RMOP-medium (preparation see below). 35 μl of a gold suspension (0.6 micron, Biorad, M0nchen; 60 mg/ml ethanol) is transferred into a sterile Eppendorf-cup (Treff, Fisher Scientific, Ingolstadt, Germany), collected by centrifugation and washed with 1 ml sterile H₂O. The gold pellet is resuspended in 230 μl sterile H₂O and 250 μl 2.5 M CaCl₂ and 25 μg DNA (transformation vectors pIC584, pIC583 and pIC582 respectively) are added. After thoroughly resuspending the mixture, 50 μl 0.1 M spermidin are added, mixed and incubated for 10 min. on ice. Then the gold is collected by centrifugation (1 min., 10000 rpm) and washed two times with 600 μl ethanol (100%, p.A.). The gold is collected by centrifugation (1 min., 10000 rpm) and finally resuspended in 72 μl ethanol (100%, p.A.). A macrocarrier is inserted in the macrocarrier-holder and 5.4 μl of the gold-suspension are applied. The bombardment is carried out with a Bio-Rad (Hercules, Calif., USA) PDS-1000/He Biolistic particle delivery system using the following parameters:

rupture disc 900 psi

helium pressure 1100 psi

vacuum 26-27 inches Hg

macrocarrier at the top level

leaf piece at the third level

6 leaf pieces are bombarded each with 5.4 μl gold suspension. After bombardment the leaf pieces are incubated for 2 days at 25° C. on RMOP-medium.

Two days after bombardment leaves were cut into small pieces (ca. 3′3 mm) and transferred to solid RMOP-medium containing 500 μg/ml spectinomycin. Leaf pieces were cut again and transferred to fresh medium after 2 weeks, then every 3 weeks until no further regenerates appeared. Green regenerates were retrieved and transferred to individual plates. The lines were subjected to repeated cycles of shoot generation by cutting small leaf pieces, which form new regenerates on RMOP-medium with 500 μg/ml spectinomycin. Rooting of selected regenerates was done on B5-medium containing 500 μg/ml spectinomycin.

| | |
|---|---|
| RMOP (pH5.8 with KOH): | |
| NH₄NO₃ | 1650 μg/ml |
| KNO₃ | 1900 μg/ml |
| CaCl₂ × 2H₂O | 440 μg/ml |
| MgSO₄ × 7H₂O | 370 μg/ml |
| KH₂PO4 | 170 μg/ml |
| EDTA-Fe(III)Na | 40 μg/ml |
| KI | 0.83 μg/ml |
| H₃BO₃ | 6.2 μg/ml |
| MnSO₄ × H₂O | 22.3 μg/ml |
| ZnSO₄ × 7H₂O | 8.6 μg/ml |
| Na₂MoO₄ × 2H₂O | 0.25 μg/ml |
| CuSO₄ × 5H₂O | 0.025 μg/ml |
| CoCl₂ × 6H₂O | 0.025 μg/ml |
| Inositol | 100 μg/ml |
| Thiamine-HCl | 1 μg/ml |
| Benzylaminopurine | 1 μg/ml |
| Naphthalene acetic acid | 0.1 μg/ml |
| Sucrose | 30000 μg/ml |
| Agar, purified | 8000 μg/ml |
| B5 (pH5.7 with KOH) | |
| KNO₃ | 2500 μg/ml |
| CaCl₂ × 2H₂O | 150 μg/ml |
| MgSO₄ × 7H₂O | 250 μg/ml |
| NaH₂PO₄ × H₂O | 150 μg/ml |
| (NH₄)₂SO₄ | 134 μg/ml |
| EDTA-Fe(III)Na | 40 μg/ml |
| KI | 0.75 μg/ml |
| H₃BO₃ | 3 μg/ml |
| MnSO₄ × H₂O | 10 μg/ml |
| ZnSO₄ × 7H₂O | 2 μg/ml |
| Na₂MoO₄ × 2H₂O | 0.25 μg/ml |
| CuSO₄ × 5H₂O | 0.025 μg/ml |
| CoCl₂ × 6H₂O | 0.025 μg/ml |
| Inositol | 100 μg/ml |
| Pyridoxine-HCl | 1 μg/ml |
| Thiamine-HCl | 10 μg/ml |
| Nicotinic acid | 1 μg/ml |
| Sucrose | 20000 μg/ml |
| Agar, purified | 7000 μg/ml |

Molecular Analysis of Potential Plastid Transformants by Southern Analysis

3 mg of total plant DNA per analysed plant are digested with the appropriate restriction enzyme and separated on a TBE-agarose gel (1%). The DNA is denatured and transferred to a positively charged nylon membrane (Hybond-N+, Amersham) as described in Ausubel et al., 1999: Short protocols in molecular biology, Wiley, 4^{th} edition, Unit 2.9A. The filter is hybridised with digoxigenin-labeled probes in DIG Easy Hyb Buffer (Roche Diagnostics GmbH, Mannheim, Germany), and hybridisation signals are detected using the DIG Luminescent Detection Kit (Roche). The membrane is exposed to a X-OMAT LS film at room temperature.

A fragment suitable for discrimination between wild type and transformed plastome is gel purified using the QIAquick Gel Extraction Kit (QIAgen, Hilden, Germany), labeled with digoxigenin using the Roche DIG DNA Labeling Kit and used for hybridisation.

### Example 2

### Construction of a Vector for Insertion of Sequences into an Existing Operon (atpE) and Plastid Transformation Therewith

The construction of plastid transformation vectors pIC500, pIC 567 and pIC574 is described in example 1.

Construction of Plasmid pIC570 (Flanking Regions atpE-Operon).

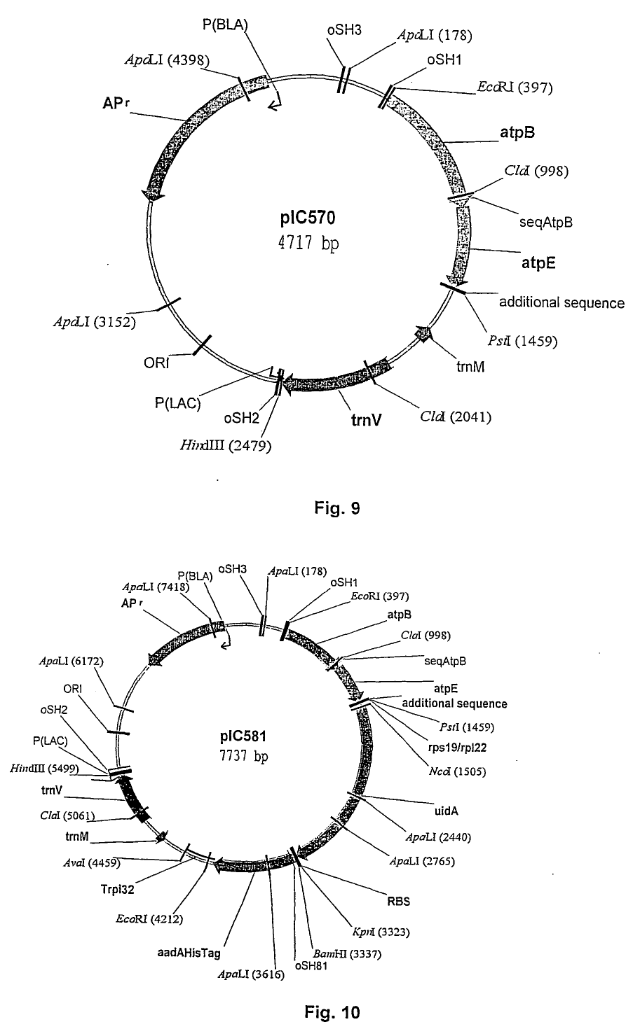

The left flanking region of the N. tabacum atpE-operon-3′-region is amplified under standard PCR conditions using 1 U Pfu-polymerase (Promega), 0.1 μM primer oSH89 (tatagggcccgaagtatcggccttattgg), 0.1 μM primer oSH90 (catgctgcagttatgaaatcggattgatagcc) and 30 ng N. tabacum DNA as template. The PCR-mixture is denatured at 95° C. for 5 min., followed by 35 cycles for 30 sec. at 95° C., 45 sec. at 50° C. and 3 min. at 72° C. Synthesis is completed by a final incubation for 7 min. at 72° C. The right flanking region of the N. tabacum atpE-operon-3′-region is amplified under standard PCR conditions using 1 U Pfu-polymerase (Promega), 0.1 μM primer oSH91 (catgctgcagttggtacgttcgaataataaaaag), 0.1 μM primer oSH92 (tatagaagcttgcattgggctctttcattaactg) and 30 ng N. tabacum DNA as template. The PCR-mixture is denatured at 95° C. for 5 min., followed by 35 cycles for 30 sec. at 95° C., 45 sec. at 50° C. and 3 min. at 72° C. The synthesis is completed by a final incubation for 7 min. at 72° C. The PCR-products are purified with the PCR-purification-kit from Qiagen resulting in 50 μl LFatpE and 50 μl RFatpE respectively. LFatpE is digested for 2.5 h at 37° C. with 40 U PstI and 40 U Bsp120I in a total volume of 100 μl of 1× Y⁺-buffer (MBI). RFatpE is digested for 2.5 h at 37° C. with 40 U PstI and 40 U HindIII in a total volume of 100 μl of 1× R⁺-buffer (MBI). Digested LFatpE and RFatpE are purified with the PCR-purification-kit from Qiagen resulting in 30 μl LFatpE-P/B and 30 μl RFatpE-P/H respectively. 10 μg of plasmid pIC500 are digested with 100 U HindIII, 100 U Bsp120I and 100 U XbaI in a total volume of 300 μl of 1× Y⁺-buffer (MBI) at 37° C. for 3 h. 5 μl alkaline phosphatase (MBI) are added and incubated for 30 min. at 37° C. The digested plasmid is purified on a 0.8% agarose gel. The vector fragment at 2640 bp is excised and purified with the Qiagen gel purification kit resulting in 50 μl pIC500-B/H/AP. 76 fmol pIC500-B/H/AP, 140 fmol LFatpE-P/B and 140 fmol RFatpE-P/H are ligated with 0.5 U T4-ligase (Promega) in 10 μl T4-ligase-buffer (Promega) at 4° C. over night. 2 μl of the ligation mixture are transformed into electrocompetent E. coli cells according to example 1. The structure of the resulting vector pIC570 is given in FIG. 9.

Construction of Plasmid pIC581 (Operon+Flanking Regions)

5 μg of pIC576 are digested for 2 h at 37° C. with 30 U PstI and 30 U Mph1103I in a total volume of 100 μl of 1× Y⁺-buffer (MBI). The digested pIC576 DNA is separated on a 0.8% agarose gel. The fragment containing Srps19+uidA+RBS+aadA+Trpl32 is excised and purified with the Qiagen gel extraction kit resulting in 30 μl Srps19+uidA+RBS+aadA+Trpl32-P/M. 1 μg pIC570 is digested for 2 h at 37° C. with 30 U PstI in a total volume of 100 μl of 1× O⁺-buffer (MBI). 1 μl of alkaline phosphatase is added to the restriction mixture of pIC570 and incubated for 30 min. Digested pIC570 is purified with the PCR-purification-kit from Qiagen resulting in 30 μl pIC570-P/AP. 60 fmol pIC570-P/AP and 60 fmol Srps19+uidA+RBS+aadA+Trpl32-P/M are ligated with 0.5 U T4-ligase (Promega) in 10 μl T4-ligase-buffer (Promega) at 4° C. over night. 2 μl of the ligation mixture are transformed into electrocompetent E. coli cells. Transformants with plasmids containing the desired insertion are identified by restriction analysis. The structure of the resulting vector pIC581 is given in FIG. 10. Plastid transformation and analysis of transformants is done as described in Example 1.

### Example 3

### Plastid Transformation Utilizing a Fragment of the 16S rDNA Conferring Spectinomycin Resistance

All cloning procedures were carried out using standard protocols (Ausubel et al., 1999: Short protocols in molecular biology, Wiley, 4^{th} edition).

For generating a fragment of the tobacco 16S rDNA conferring spectinomycin resistance, the 3′-part of the 16S rDNA (pos. 102796 to 104274) (Accession Number Z00044) was amplified with PCR from isolated tobacco (N. tabacum cv. Petite Havana) DNA using primers 16S-li (5′-gctggcggcatgcttaacac-3′) and 16S-re (5′-ccagcatgcattagctctccctg-3′). Primer 16S-re introduces two base exchanges into the rm16-tml spacer region to create an SphI restriction site. PCR reactions were done in 100p1 volumes with Pfu polymerase (Promega Corporation, Madison, USA) using conditions recommended by Promega in a Hybaid PCR cycler for 35 cycles of 95° C./30 sec, 57° C./30 sec; and 72° C./3 min. The resulting fragment was cut at both ends with SphI and ligated into the SphI-site of cloning vector pIC500 (see example 1). Clones in which the 3′-end of the rm16 sequence was located next to the KpnI-site were selected. For introducing a point mutation conferring spectinomydin resistance (Svab and Maliga, 1991: A to C at position 103898 according to tobacco plastome positions, Accession Number Z00044), a 838 bp AccIII-BsrGI fragment was replaced by the corresponding fragment from plasmid pUC19-Spec (see example 4), in which this mutation is present.

As a second flank for homologous recombination, tobacco plastid DNA sequence 104273 to 105269 was amplified by PCR from isolated tobacco (N. tabacum cv. Petite Havana) DNA, using primers FLR-li (5′-cctctagagggtattttggtttgacactgc-3′) and FRL-re (5′-agctgcagtccaaccaattgggagagaatc-3′; PCR conditions as above). The resulting fragment was cut at both ends with XbaI and PstI and ligated into the corresponding restriction sites of the plasmid described above, resulting in plasmid pICspecTF. The construct was sequenced to prove the correct sequences of the regions containing plastid DNA.

For insertion of a reporter sequence into pICspecTF, the uidA sequence linked to a plastid ribosomal binding site was excised from plasmid pIC582 (see example 1) with PstI and KpnI, treated with T4 DNA polymerase, and ligated into the T4 DNA polymerase treated NotI-site of plasmid pICspecTF to give plasmid pIC588 (see FIG. 11). The site of insertion of the foreign sequence in plastid DNA after transformation is downstream of the rm16 sequence, within the rm16-operon.

Plastid transformation of tobacco with construct pIC588 is made as described in example 1. Selection of regenerates containing transformed plastids is made on RMOP-medium with 500 μg/ml spectinomycin.

### Example 4

### Plastid Transformation with Vector pIC587, Allowing Integration of Foreign Sequences at Two Different Sites

All cloning procedures were carried out using standard protocols (Ausubel et al., 1999: Short protocols in molecular biology, Wiley, 4^{th} edition).

For generating a fragment of the tobacco 16S rDNA conferring spectinomycin and streptomycin resistance, the 3′-part of the 16S rDNA (pos. 103261 to 104121) (Accession Number Z00044) was amplified with PCR from isolated tobacco (N. tabacum cv. Petite Havana) DNA using primers rm16-li (5′-tccggaatgattgggcgtaaa-3′) and rm16-re (5′-ggcggtgtgtacaaggcccg-3′). PCR reactions were done in 100 μl volumes with Pfu polymerase (Promega Corporation, Madison, USA) using conditions recommended by Promega in a Hybaid PCR cycler for 35 cycles of 95° C./30 sec, 57° C./30 sec, and 72° C./3 min. The resulting fragment was ligated into the SmaI-site of cloning vector pUC19 (pUC19-rm16Frag.).

For introducing a point mutation (C to T) at position 103899 (according to tobacco plastome positions, Z00044) conferring spectinomycin resistance (Svab et al., 1990) the complete plasmid was amplified by inverse PCR with 5′-phosphorylated mutation primers spec1 (5′-agtaggagtggaaggaggcc-3′) and spec2 (5′-acagttcagtagtacgggga-3′; PCR conditions as above, but elongation time 7 min), purified, and ligated to yield plasmid pUC19-Spec.

For introducing a point mutation (C to A) at position 103620 (according to tobacco plastome positions, Z00044) conferring streptinomycin resistance (Svab et al., 1990) the complete plasmid was amplified by inverse PCR with 5′-phosphorylated mutation primers strep1 (5′-tcaaagtaagaacgcttgca-3′) and strep2 (5′-ttttccttaactgcccccgg-3′; PCR conditions as above, but elongation time 7 min), purified, and ligated to yield plasmid pUC19-Strep. The constructs were sequenced to prove the correct sequences of the regions containing plastid DNA.

For construction of a fragment carrying both point mutations, a 460 bp ApaI-BsrGI fragment from plasmid pUC19-Strep was replaced by the corresponding fragment from plasmid pUC19-Spec, to give plasmid pUC19-Strep-Spec.

Plastid transformation vector pIC586 was constructed in the following way: The aadA coding sequence from plasmid pIC582 was excised with SacI and KpnI, the plasmid was treated with T4 DNA polymerase, and ligated.

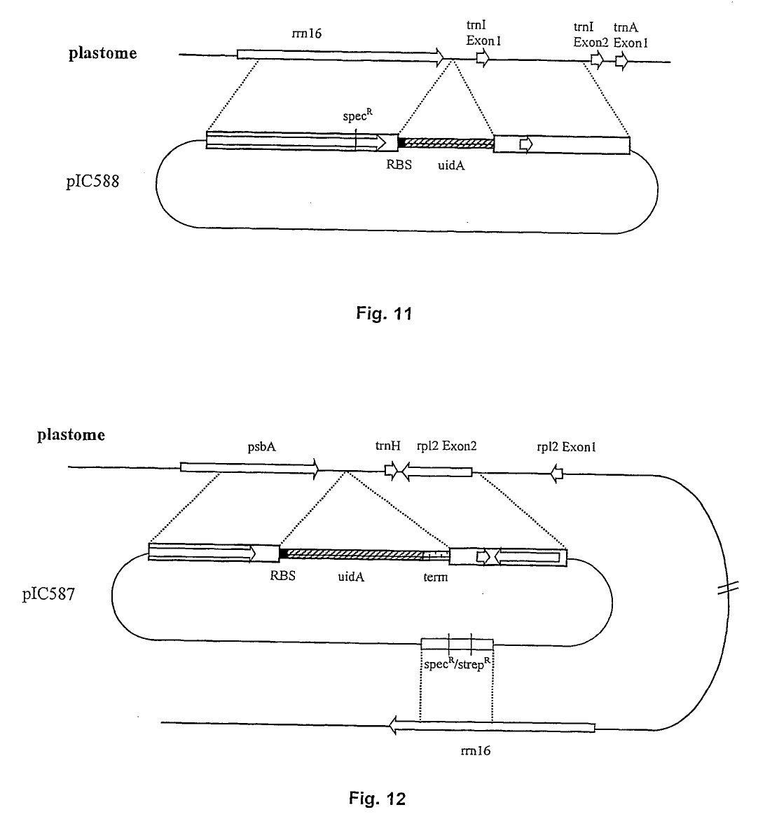

For insertion of the rm16 fragment with both point mutations leading to spectinomycin and streptomycin resistance into pIC586, the fragment was excised from pUC19-Strep-Spec with AccIII and BsrGI, treated with T4 DNA polymerase, and ligated into the T4 DNA polymerase treated AatII-site of plasmid pIC586 to give plasmid pIC587 (see FIG. 12).

Plastid transformation of tobacco with construct pIC587 is made as described in example 1. Selection of regenerates containing transformed plastids is made on RMOP-medium with 500 μg/ml spectinomycin.

### Example 5

### Repeated Plastid Transformations by Reciprocal Exchange of Spectinomycin and Streptomycin Resistance

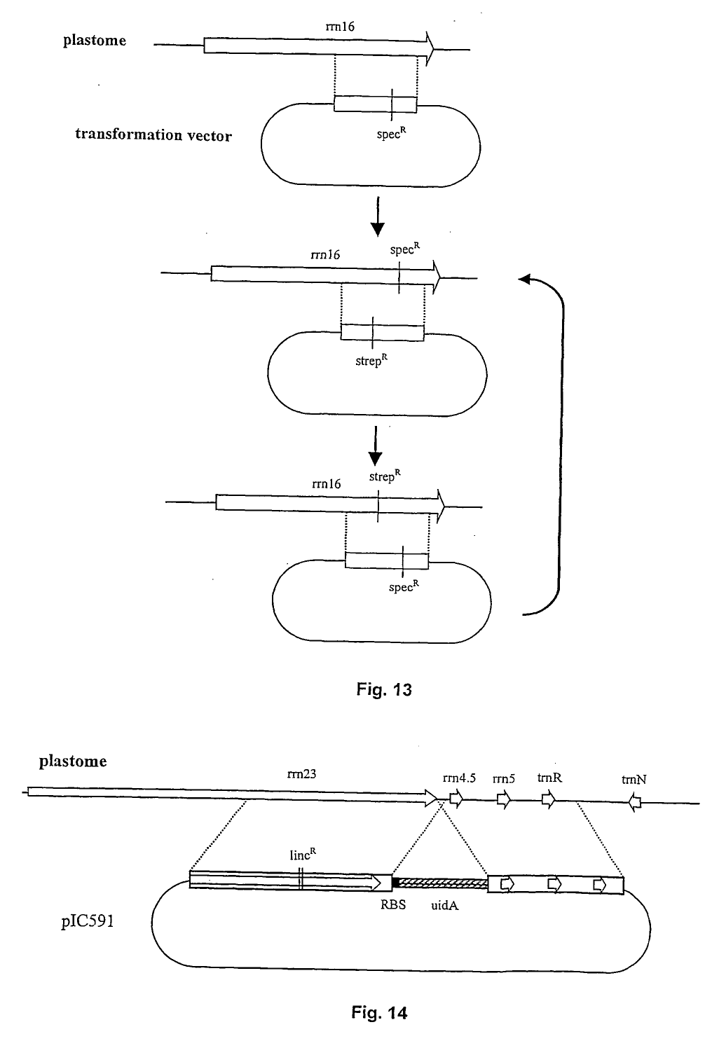

Plastid transformation of tobacco plants as described in example 1 with transformation vector pUC19-Spec (see example 4) leads to spectinomycin-resistant plants carrying a point mutation in the rm16 sequence. The resulting transplastomic plants are subsequently transformed with vector pUC19-Strep (see example 4), and selection is made on 500 μg/ml streptomycin. Homologous recombination between plastome and transformation vector leads to introduction of the point mutation conferring streptomycin resistance, while the mutation conferring spectinomycin resistance is removed concomitantly. Successful reintroduction of sensitivity to spectinomycin is tested by a 10 day exposure of plants or leaf pieces to spectinomycin, which leads to bleaching of the leaves, but does not kill the plants, which can therefore be further cultivated when put back on medium without spectinomycin. Transplastomic plants resulting from the second transformation can subsequently be transformed with pUC19-Spec again, with spectinomycin as selection agent; homologous recombination leads to reintroduction of the point mutation conferring spectinomycin resistance, while streptomycin resistance is removed. This method allows several plastid transformations of a plant by alternating exchange of the two antibiotic resistances. Additional sequences of interest can be introduced with the same vectors, or by cotransformation with any plastid transformation vector. For a schematic description of the repeated plastid transformation process see FIG. 13.

### Example 6

### Plastid Transformation Utilizing a Fragment of the 23S rDNA Conferring Resistance to Lincomycin

All cloning procedures were carried out using standard protocols (Ausubel et al., 1999: Short protocols in molecular biology, Wiley, 4^{th} edition).

For generating a mutant version of the tobacco 23S rDNA conferring lincomycin resistance, the 3′-part of the 23S rDNA (pos. 107700 to 109223) (Accession Number Z00044) was amplified with PCR from isolated tobacco (N. tabacum cv. Petite Havana) DNA using primers 105 (5′-AGGCATGCAAACTTCTGTCGCTCCATCC-3′) and 106 (5′-AGGCATGCTMGATCAGGCCGAAAGGC-3′). PCR reactions were done in 100 μl volumes with Pfu polymerase (Promega Corporation, Madison, USA) using conditions recommended by Promega in a Hybaid PCR cycler for 35 cycles of 95° C./30 sec, 55° C./30 sec, and 72° C./3 min. The resulting fragment was cut at both ends with SphI and ligated into the SphI-site of cloning vector pIC500 (see example 1). Clones in which the 3′-end of the 23S sequence was located next to the KpnI-site were selected. For introducing point mutations conferring lincomycin resistance (Cséplö et al., 1988: positions 108375, 108401 and 108402 according to tobacco plastome, Z00044), the complete plasmid was amplified by inverse PCR with 5′-phosphorylated mutation primers 82 (5′-GTCCATCAGGCGTAAAAGTGTCTGTACAGATAAAG-3′) and 104 (5′-GTGGACCTGTCCCTCTGGGATACTTCGAAG-3′; PCR conditions as above, but elongation time 7 min), purified, and ligated to yield plasmid pIClinc.

As a second flank for homologous recombination, tobacco plastid DNA sequence 109139 to 110151 was amplified by PCR from isolated tobacco (N. tabacum cv. Petite Havana) DNA, using primers 107 (5′-CCTCTAGATTCCGACTTCCCCAGAGCC-3′) and 108 (5′-ACCTGCAGACAAAAGACCCACACCCAAG-3′; PCR conditions as above, elongation time 3 min). The resulting fragment was cut at both ends with XbaI and PstI and ligated into the corresponding restriction sites of plasmid pIClinc, resulting in pIClincTF. The construct was sequenced to prove the correct sequences of the regions containing plastid DNA.

For insertion of a reporter sequence into pIClincTF, the uidA coding sequence linked to a plastid ribosomal binding site is excised from plasmid pIC582 (see example 1) with PstI and KpnI, treated with T4 DNA polymerase, and ligated into the T4 DNA polymerase treated NotI-site of plasmid pIClincTF to give plasmid pIC591 (see FIG. 14). The site of insertion of the foreign sequence in plastid DNA after transformation is downstream of the rm23 sequence, within the rm16-operon.

Plastid transformation of tobacco with construct pIC591 is made as described in example 1. Selection of regenerates containing transformed plastids is made on RMOP-medium with 1 to 3 mg/ml lincomycin.

### Example 7

### Plastid Transformation Utilizing a Fragment of the psbA Gene Conferring Atrazine Resistance

All cloning procedures were carried out using standard protocols (Ausubel et al., 1999: Short protocols in molecular biology, Wiley, 4^{th} edition). Vector pIC582 (see example 1) was used as starting vector. It contains part of the psbA gene in the right flanking fragment. The specific point mutation (first base of codon 264 is changed from AGT [serine] to GGT [glycine], Sato et al., 1988) in the psbA coding sequence that leads to resistance to atrazine was introduced by PCR. One primer was designed containing the specific point mutation and the two other base exchanges that generate a new restriction site without changing the amino acid sequence of the encoded D1 protein. Primers ‘oSK109-pm-atrazine’ (tactttcaacaactcgcgatcgttacacttcttcc) and oSH85 (catgctgcagcaagaaaataacctctccttc) were used to amplify a fragment of about 450 bp. The resulting fragment (containing the point mutation for atrazine resistance and the new restriction site, NruI) and primer oSH84 (tatagggcccagctataggtttacatttttaccc) were used for a second PCR to amplify the whole mutagenized right flanking sequence. The wildtype fragment without the mutations was replaced by the new mutagenized PCR fragment. The construct was sequenced to prove correct sequence and introduction of the mutations.

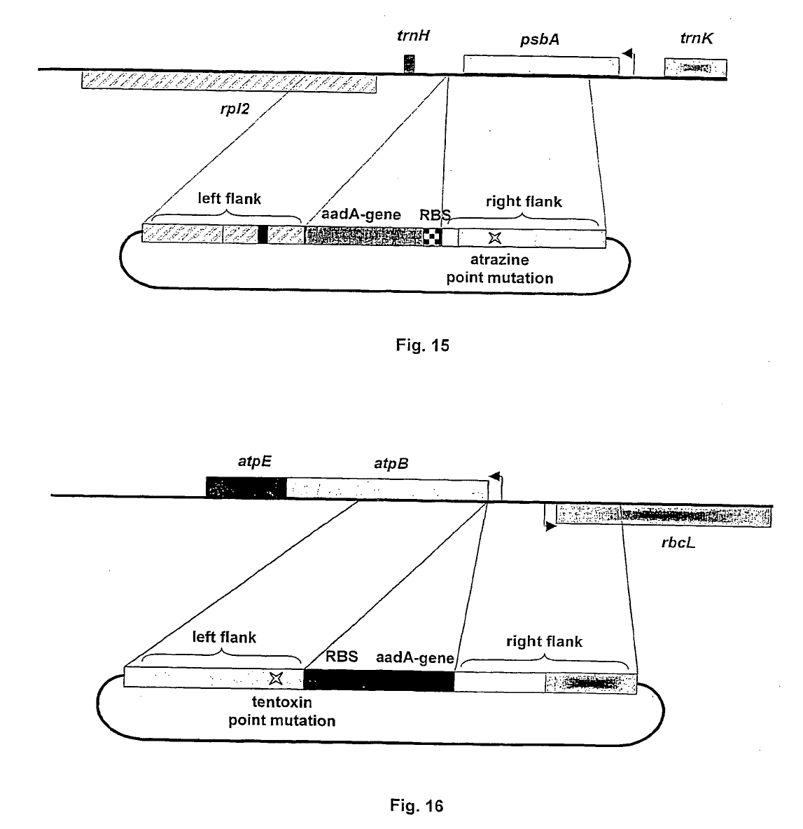

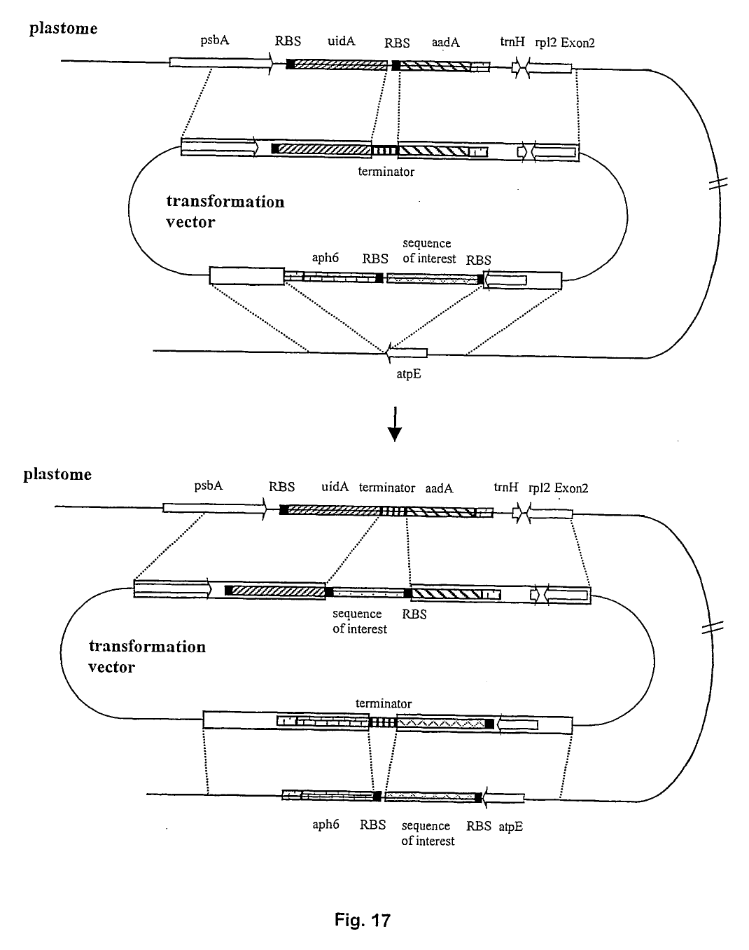

Plastid transformation of tobacco with construct pIC592 (FIG. 15) can be made as described in example 1. Selection of regenerants containing transformed plastids can be made on RMOP-medium with reduced sucrose concentration (0.3%) and 50-100 μM atrazine (Sato et al., 1988).

### Example 8

### Plastid Transformation Utilizing a Fragment of the atpB Gene Conferring Tentoxin Resistance

All cloning procedures were carried out using standard protocols (Ausubel et al., 1999: Short protocols in molecular biology, Wiley, 4^{th} edition).

For generating a fragment of the tobacco atpB coding sequence conferring tentoxin resistance, first a part of atpB (left flanking region, about 1 kb from the start codon) is amplified by PCR as well as the right flanking region (containing about 1 kb of the atpB 5′ region). For PCR amplification of the left flank primers oSK . . . -fatpB3'hs (gggaattccatatgagaatcaatcctactacttct) and oSK . . . -ratpB3'hs (aaaactgcagaatcgtctgcgggtacataaac) are used, generating two new restriction sites at the fragment ends (NdeI and PstI). For PCR amplification of the right flank primers oSK . . . -fatpB5'hs (agtcgagctcatgtaccagtagaagattcg) and oSK . . . -ratpB5'hs (cgggatccaataataaaataaataaatatgtcgaaa) are used, generating two new restriction sites (SacI and BamHI) at the ends as well.

The first cloning step is the ligation of the NdeI/PstI digested left flank into pUC19. The specific point mutation (third base of codon 83 is changed from GAC [asparagine] to GAG [glutamine]; Avni et al., 1992; Hu et al., 1997) is introduced into this fragment by PCR using primers oSK . . . -fatpB3'hs, oSK . . . -ratpB3'hs and oSK . . . -pm-tentoxin: primer oSK . . . -pm-tentoxin (ctctgtagcgctcatagctaca) contains specific base exchanges leading to tentoxin resistance and a new restriction site (Eco47III). The mutations for generating the Eco47III site do not change the amino acid sequence of the atpB gene product. In a first amplification step a fragment of about 250 bp is amplified using primers oSK . . . -fatpB3'hs and oSK . . . -pm-tentoxin. This fragment and primer oSK . . . -ratpB3'hs are used as primers for a second PCR to amplify the whole mutagenized left flanking sequence.

All three fragments (right flank, mutagenized left flank, and a sequence of interest) are digested with the corresponding enzymes and ligated into vector pUC19 in one step leading to plastid transformation vector pIC593 (FIG. 16). Sequencing analysis are showing the corrrect sequences of the PCR amplified and mutagenized flank(s).

An important step is to ligate an artificial ribosomal binding site (RBS, vector ‘pUC16S aadA Sma vollst’, Koop et al., 1996) downstream of the sequence of interest (e.g. aadA, FIG. 16) to have a RBS for the translation start of the atpB coding region. After this cloning step the sequence of interest and the RBS can be cut out as one fragment and cloned into pUC19 together with the flanks. This cloning procedure was performed by cloning the aadA coding sequence also upstream to the RBS into ‘pUC16S aadA Sma volist’ plasmid. Then a fragment containing both the aadA sequence and the RBS can be cut out of this vector and cloned together with the right/left flanks into pUC19 as described above.

Plastid transformation with construct pIC593 (FIG. 16) can be made as described in example 1. As target plant Nicotiana plumbaginifolia can be used which is sensitive to tentoxin (in contrast to Nicotiana tabacum which is naturally tentoxin resistant). Selection of regenerants containing transformed plastids can be made on RMOP-medium with reduced sucrose concentration (0.3%; Cséplö et al., 1986) and about 10-20 μg/ml tentoxin (Avni and Edelman, 1991).

### Example 9

### Plastid Transformation Utilizing a Fragment of the psbA Gene Conferring Metribuzin Resistance

All cloning procedures were carried out using standard protocols (Ausubel et al., 1999: Short protocols in molecular biology, Wiley, 4^{th} edition).

Examples 1 and 7 describe in detail how a specific point mutation in the psbA coding sequence can be used as selection marker for plastid transformation. Numerous specific point mutations in the psbA coding sequence can be used as selection markers for plastid transformation as described in the literature (for review see: Hock and Elstner, 1995). Another example is the use of metribuzin as selection agent. As described in Schwenger-Erger et al., 1993, and Schwenger-Erger et al., 1999 several point mutations (two or three changes in the amino acid sequence between positions 219 and 272; amino acid exchange at the position 184 [ile→asn]) in the psbA coding sequence leads to resistance to metribuzin in Chenopodium rubrum. These mutations are in a very conserved region of the plastome. Therefore, the same point mutations should confer resistance also to tobacco and other sensitive plants.

Cloning strategy and introduction of the point mutation are carried out in the same way as described in example 7.

Plastid transformation of tobacco with construct pIC594 will be performed as described in example 1. Selection of regenerates containing transformed plastids can be made on RMOP-medium with reduced sucrose concentration (0.3%) and 0,01-1,0 μM metribuzin (final resistance concentration: 1-10 μM; Schwenger-Erger et al., 1993).

### Example 10

### Plastid Transformation Utilizing a Fragment of the psbA Gene Conferring Diuron Resistance

All cloning procedures were carried out using standard protocols (Ausubel et al., 1999: Short protocols in molecular biology, Wiley, 4^{th} edition).

In example 1 and example 7 it is described in detail how a specific point mutation in the psbA coding sequence can be used as selection marker for plastid transformation. Numerous specific point mutations in the psbA coding sequence can be used as selection markers for plastid transformation as described in the literature (for review see: Hock and Elstner, 1995). Another example is the use of diuron as selection agent. As described in Wolber et al., 1986 a point mutation in valine-219 leads to diuron resistance in C. reinhardtii. The same point mutation that leads to atrazine resistance in tobacco also confers cross-resistance to diuron (Sato et al., 1988; first base of codon 264 is changed from AGT [serine] to GGT [glycine]).

Cloning strategy and introduction of the point mutation are carried out in the same way as described in example 7.

Plastid transformation of tobacco with construct pIC595 will be performed as described in example 1. Selection of regenerates containing transformed plastids can be made on RMOP-medium with reduced sucrose concentration (0.3%) and 1-5 μM diuron (20-fold higher diuron resistance compaired with the wildtype Sato et al., 1988).

### Example 11

### Plastid Transformation Using Regulatory Elements for Inactivation of Selection Markers

This example shows a possibility of “recycling” of selection markers by inactivation of the marker through exchange of regulatory elements.

The example is based on a plant which has been transformed with vector pIC582 and therefore has the aadA marker and an additional sequence of interest inserted in the psbA operon. In a subsequent transformation, a further sequence of interest is introduced at another locus, such as the atpE operon, using a transformation vector based on pIC581, but with the selection marker aph6, which confers kanamycin resistance (Bateman and Purton, 2000), instead of aada; aph6 has been used as a selection marker for plastid transformation in Chlamydomonas reinhardtii, and its use in higher plants is at present optimized in our laboratory. Simultaneously, aadA expression is suppressed by introducing a sequence comprising strong termination activity, such as trnS (Stern and Gruissem, 1987), upstream of the aadA coding sequence, using a transformation vector based on pIC582, where the RBS upstream of aadA is replaced by said terminator element. The two transformation cassettes are preferably on a single plasmid, as described in example 5. As the resulting transgenic plant is not resistant to spectinomycin anymore, spectinomycin selection can be used for a further transformation, in which aadA expression can be restored by exchanging the terminator element against an RBS element again; an additional sequence of interest can be introduced upstream of it. Simultaneously, kanamycin resistance can be removed by the strategy described, opening the way for further transformations.

### Example 12

### Insertion of a Modified Intein into psbA

The protein splicing element (intein) of the vacuolar ATPase subunit (VMA) of Saccharomyces cerevisiae is modified by exchanging the original endonuclease domain by the selection marker aadA. This modified intein is inserted into the tobacco plastid psbA open reading frame. After integration into the plastome aadA is excised posttranslationally from the expressed psbA gene yielding a functioning selection marker inside of the plastids.

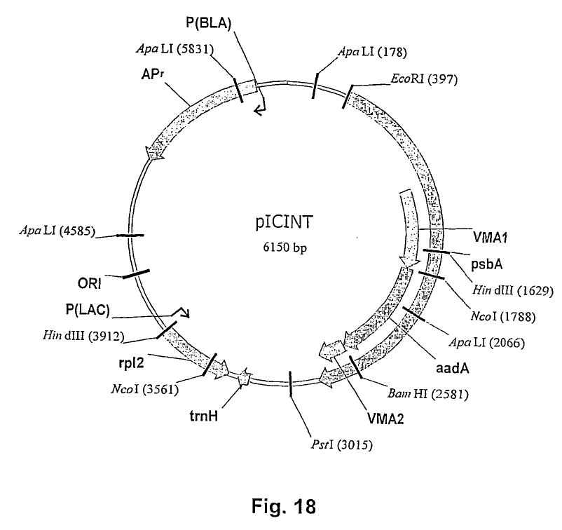

The 5′-part of the VMA intein is amplified by PCR under standard conditions using oligos 5′-CATCTAGACTGCTTTGCCAAGGGTACCAATG-3′ and 5′-GACCATGGAACCTTCAATGGTGAGATGAAAC-3′ and S. cervisiae DNA as template. The resulting PCR-product of 630 bp is purified and digested with XbaI and NcoI yielding VMA1. The 3′-part of the VMA intein is amplified by PCR under standard conditions using oligos 5′-GAGGATCCTGGAGATGTTTTGCTTAACG-3′ and 5′-GATCTAGACAATTATGGACGACAACCTGG-3′ and S. cervisiae DNA as template. The resulting PCR-product of 220 bp is purified and digested with XbaI and BamHI yielding VMA2. The aadA gene is amplified by PCR under standard conditions using oligos 5′-ATGCCATGGCTCGTGAAGCGG-3′ and 5′-GAGGATCCTTGCCAACTACCTTAGTG-3′ and pIC519 as template. The resulting PCR-product of 804 bp is purified and digested with NcoI and BamHI yielding aadAI. Vector pIC569 is digested with XbaI, dephosphorylized with alkaline phosphatase and ligated with the restricted fragments VMA1, aadAI and VMA2 yielding vector pICINT (FIG. 18). Plastid transformation of tobacco with vector pICINT and selection of regenerates is performed as described in example 1.

### Example 13

### Creation of a Fusion Protein of Plastidic YCF9 and Heterologous GUS

The uidA gene is fused to the plastidic ycf9 gene to generate a fusion protein under control of ycf9 regulatory elements. For selection a point mutation in the rm16 gene is generated as described in example 4.

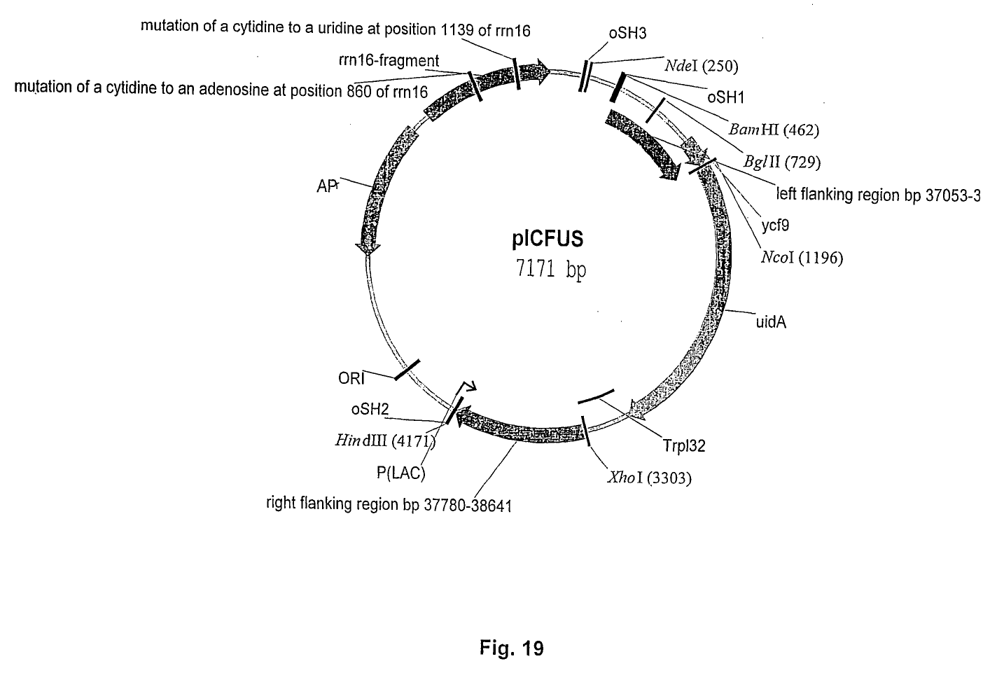

The vector backbone including the rm16 point mutation is generated by PCR amplification of the vector backbone of pIC587 under standard conditions using oligos 5′-CAGGATCCACTGGCCGTCGTTTTAC-3′ and 5′-GATGAAGCTTGGTCATAGCTGTTTCCTG-3′. The resulting PCR product of 3474 bp is purifid and digested with BamHI and HindIII resulting in pICL. The tobacco plastome sequence from bp 37053 to bp 37780 is amplified by PCR under standard conditions with oligos 5′-CAGGATCCGGTGGGATAGCCGAGCC-3′ and 5′-GACCATGGAAGAGATGAGAGAATTAAGG-3′ using tobacco plastome DNA as template. The resulting PCR product is purifid and digested with BamHI and NcoI resulting in LFycf9. The tobacco plastome sequence from bp 37780 to bp 38641 is amplified by PCR under standard conditions with oligos 5′-CTACTCGAGTGAACCTATTCGTCGGAGACC-3′ and 5′-GATGAAGCTTCACATACTTCGTGAAATGGTTC-3′ using tobacco plastome DNA as template. The resulting PCR product is purifid and digested with HindIII and XhoI resulting in RF. The uidA gene is cut out of pIC587 with NcoI and XhoI. The restriction products are purified on an agarose gel and the band at 2107 bp is collected and purified yielding in RuidA. The fragments pICL, LFycf9, RuidA and RF are ligated with T4-ligase yielding pICFUS (FIG. 19) and transformed into E. coli. DNA from the transformants is isolated and analysed by restriction analysis. Correct pICFUS is used for plastid transformation of tobacco and selection of regenerates is performed as described in example 4.

### Example 14

### Insertion of a Silent Gene Fragment, which is Activated in a Second Transformation Step. A uidA-Expression Cassette is Inserted in the Second Step

A fragment of a aphA6 gene, which is devoid of 5′-regulatory elements is inserted into the tobacco plastome in a first step of transformation. Selection for resistance against Spectinomycin and Streptomycin is achieved by co-integration of a marker gene fragment containing point mutations. The marker gene fragment and the aphA6 fragment are inserted into separated sites of the plastome. In a second transformation step a uidA reporter gene, which carries a spacer fragment instead of a terminator element is inserted upstream of the aphA6 fragment. By the insertion the previously silent aphA6 fragment is activated thus enabling a selection on resistance for Kanamycin.

Plasmid pIC587 is digested with Hind III and Nde I and separated on an agarose gel. The 3261 bp vector fragment, which carries a fragment of a marker, is excised from the gel and purified. Using total DNA from tobacco as a template a 1000 bp fragment from the tobacco plastome (position 114.700-115700) is amplified under standard PCR conditions with the oligos PrF1: 5′-CGAAGCTTTTTTCTTTTTATTAAGTTCA-3′ and PrF2: 5′-CATATGCCTGCAGGTATATAATAATTCAGAGAAA-3′. The primers introduce HindIII, NdeI and SdaI restriction sites. The purified 1062 bp PCR fragment is digested with Hind III and NdeI and again purified. The Hind III and Nde digested pIC587vector fragment is ligated with the Hind III and Nde digested PCR fragment and transformed into bacteria yielding plasmid pIC587-rpl32. The cloned plasmid is digested with SdaI and XmaI. The approximately 4280 bp vector fragment is excised from a gel and purified. A 1303 bp NdeI/XbaI fragment is isolated from the plasmid pSK.KmR (Bateman and Purton, 2000) using enzyme digestion and agarose gel purification. The fragment comprises the coding region of the aphA6 gene and a 3′-sequence from the Chlamydomonas reinhardtii rbcL gene.

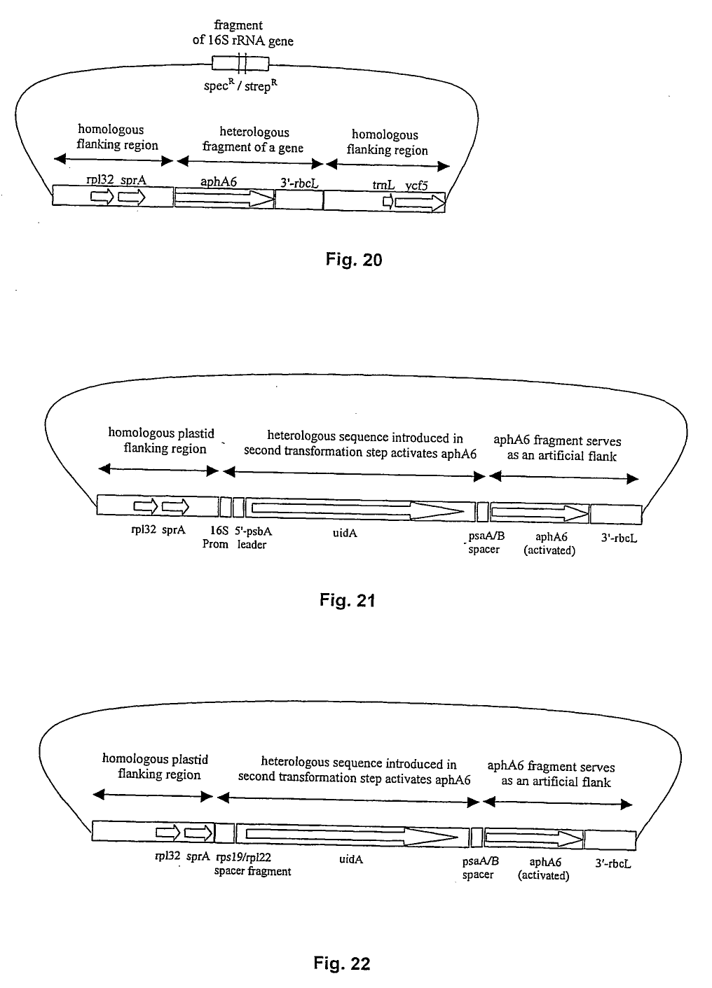

Using total DNA from tobacco as a template a 1000 bp fragment from the tobacco plastome (position 115701-116700) is amplified under standard PCR conditions with the oligos PrF3: 5′-GTCTAGAAGATACCCATGTATATCTTG-3′ and PRF4: 5′-GCCCGGGAGTTAAAATACCTGACGTAGC-3′. The primers introduce XbaI and XmaI restriction sites. The purified 1054 bp PCR fragment is digested with XbaI and XmaI and purified again. The aphA6 fragment and the PCR fragment are ligated with the excised SdaI/XmaI pIC587-rpl32 vector fragment and transformed into bacteria yielding the plastid transformation vector pIC-aphA6-rpl32 (FIG. 20). Plastid transformation of tobacco with vector pIC-aphA6-rpl32 and selection of regenerates on Spectinomycin and/or Streptomycin containing media is performed as described in example 1.

Transplastomic plants are recovered and analyzed for the correct integration of the aphA6 gene fragment.

The plasmid pIC587-rpl32 is digested with SdaI and XmaI. The approximately 4280 bp vector fragment is excised from a gel and purified. Using plasmid psb::rbc (Eibl et al, 1999) as a template an approximately 2040 bp fragment is amplified under standard PCR conditions with the primers PrGus1: 5′-GCCTGCAGGGCCGTCGTTCAATGAGAATG-3′ and PrGus2: 5′-GCCCGGGTTAATTAATCATTGTTTGCCTCCCTGCT-3′. The primers introduce SdaI, PacI and XmaI restriction sites. The amplified fragment contains a plastid 16S promotor fragment, 5′-psbA leader fragment and the coding region of the uidA gene. The PCR fragment is digested with SdaI and XmaI, purified and ligated to the SdaI and XmaI digested pIC587-rpl32 vector fragment. The ligation reaction is transformed into bacteria yielding plasmid pIC587-rpl32-GUS. Plasmid pIC587-rpl32-GUS is digested with PacI and XmaI. The approximately 6300 bp vector fragment is gel purified. Oligo OpsaA/B (5′-TTAATTAATGGCTAGGAGGATTTGAAAAGCATTCATATGCCG-3′, containing a PacI restriction site at the 5′-end and a NdeI-site near the 3′-end) is ligated as a single strand molecule to the vector fragment. The oligo contains the spacer region between the plastid psaA and psaB genes. The ligation reaction is gel purified and treated with Taq polymerase and dNTP for 10 minutes at 72° C. to synthesize the second strand. The reaction is purified and the linear fragment is digested with NdeI. A 1303 bp NdeI/XmaI fragment is isolated from the plasmid pSK.KmR (Bateman and Purton, 2000), purified by electrophoresis and ligated to the above described linear Nde I digested vector fragment. The ligation is transformed into bacteria yielding the plastid transformation vector pIC-uidA-aphA6 (FIG. 21).

Leaves from plants containing the desired plastome modifications from transformation step 1 (using vector pIC-aphA6-rpl32) are used as a material for the second transformation step.

Plastid transformation of tobacco with vector pIC-uidA-aphA6 and selection of regenerates on Kanamycin containing media is performed as described in example 1. Positively tested plastome transformants show expression of the uidA reporter gene.

### Example 15

### Example: Insertion of a Silent Gene Fragment, which is Activated in a Second Transformation Step. A Gene Fragment Containing the Coding Region of uidA is Inserted in the Second Step

Similar as in example 14, a fragment of a aphA6 gene which is devoid of 5-regulatory elements is inserted into the tobacco plastome in a first step of transformation. Selection for resistance against Spectinomycin or Streptomycin is achieved by co-integration of a marker gene fragment containing point mutations. The marker gene fragment and the aphA6 fragment are inserted into separated sites of the plastome. In a second transformation step, a fragment of a uidA reporter gene which carries a spacer fragment instead of a terminator element is inserted upstream of the aphA6 fragment with vector pIC-uidA-aphA6-frag (FIG. 22). In contrast to example 14, the uidA fragment is devoid of a promotor.

A 53 bp plastid spacer fragment originating from the non-coding sequence between the rps19 and rpl22 gene (position 86.403-position 86.351) is fused to the 5′-end of uidA coding region. The uidA fragment and the aphA6 fragment is transcribed via the endogenous sprA promotor, thus generating an artificial operon consisting of three cistrons (sprA, uidA and aphA6). The artificial operon activates' the previously silent aphA6 fragment thus enabling a selection on resistance to Kanamycin.

### REFERENCES

Ausubel (ed.), Current Protocols in Molecular Biology, John Wiley and Sons, Inc. (1999).

Ausubel et al., 1999: Short protocols in molecular biology, Wiley, 4^{th} edition;

Avni and Edelman, 1991 MGG 225: 273-7;

Avni et al., 1992 Science 28;257(5074):1245-7;

Bateman and Purton, Mol Gen Genet (2000) 263: 404-410;

Boynton J. E., Gillham N. W., Harris E. H., Hosler J. P., Johnson A. M., Jones A. R., Randolph-Anderson B. L., Robertson D., Klein T. M., Shark K. B., et al. (1988), Science, 240, 1534-1538;

Carrer H., Hockenberry T. N., Svab Z., Maliga P. (1993), Mol. Gen. Genet., 241, 49-56.

Cséplö et al., 1986: Nuclear Techniques and In Vitro Culture of Plant Improvement: EA (ed.), Vienna. IAEA, 1986; pp. 137-146;

Cséplö et al., 1988 Mol. Gen. Genet. 214: 295-299;

Daniell H., 1999, Trends Plant Sci., 4, 467-469;

Eibl et al., 1999, Plant J., 19, 333-345;

Galvin S. B., 1998, Curr. Opin. Biotechnol., 9, 227-232.

Gray M. W., Origin and Evolution of Plastid Genomes and Genes, in: Bogorad L. and Vasil I. K. (eds.), Cell Culture and Somatic Cell Genetics of Plants, Volume 7A, Academic Press, San Diego, 1991;

Hefferon et al, 2000, Arch. Virol., 145, 945-956;

Heifetz, 2000, Biochimie, 82, 655-666;

Hock and Elstner, 1995: Schadwirkung auf Pflanzen; Spektrum, 3. Auflage; pages 155-186

Hu et al., 1997 J. Biol. Chem 272: 5457-5463;

Kavanagh T. A., Thanh N. D., Lao N. T., McGrath N., Peter S. O., Horvath E. M., Dix P. J., Medgyesy P., 1999, Genetics, 152, 1111-1122;

Koop et al., 1996, Planta, 199: 193-201;

Maniatis, E. F. Fritsch and J. Sambrook, Molecular Cloning: A Laboratory Manual, Cold Spring Harbor laboratory, Cold Spring Harbor, N.Y. (1989);

Marechal-Drouard L., Kuntz M., Weil J. H., tRNAs and tRNA Genes of Plastids, in: Bogorad L. and Vasil I. K. (eds.), Cell Culture and Somatic Cell Genetics of Plants, Volume 7A, Academic Press, San Diego, 1991;

Palmer J. D., Plastid Chromosomes: Structure and Evolution, in: Bogorad L. and Vasil I. K. (eds.), Cell Culture and Somatic Cell Genetics of Plants, Volume 7A, Academic Press, San Diego, 1991;

Sato et al., 1988 MGG 214: 358-360;

Sato et al., Mol Gen Genet Oct;214(2):358-60;

Shinozaki K., Ohme M., Tanaka M., Wakasugi T., Hayashida N., Matsubayashi T., Zaita N., Chunwongse J., Obokata J., Yamaguchi-Shinozaki K., Ohto C., Torazawa K., Meng B. Y., Sugita M., Deno H., Kamogashira T., Yamada K., Kusuda J., Takaiwa F., Kato A., Tohdoh N., Shimada H. and Sugiura M. (1986), EMBO J. 5, 2043-2049.

Schwenger-Erger et al., 1993 FEBS Lett Aug 23;329(1-2):43-6;

Schwenger-Erger et al., 1999 Z Naturforsch [C] Nov;54(11):909-14;

Shinozaki & Sugiura, 1982, Gene, 20, 91-102;

Sigalat et al., 1995, FEBS Lett 368: 253-6;

Silhavy, M. L. Berman, and L. W. Enquist, Experiments with Gene Fusions, Cold Spring Harbor Laboratory, Cold Spring Harbor, N.Y. (1984);

Staub J. M. & Maliga P., 1994, Proc. Natl. Acad. Sci. USA, 91, 7468-7472;

Staub J. M. & Maliga P., 1995, Plant J., 7, 845-848;

Stern & Gruissem, 1987, Cell, 51, 1145-1157;

Sugita M., Svab Z., Maliga P., Sugiurra M. (1997) Mol Gen Genet 257, 23-27.

Sugiura, 1991, in: Cell Culture and Somatic Cell Genetics of Plants, Vol. 7A, 125-137;

Svab, Z. et al. (1990) Proc. Natl. Acad. Sci. USA 87, 8526-8530;

Svab and Maliga, 1991; Mol. Gen Genet. 228: 316-319;

The Arabidopsis Genome Initiative, 2000, Nature, 408, 796-815;

Vera A, Sugiura M. (1994) EMBO J 13, 2211-2217.

Wolber et al., 1986; Arch Biochem Biophys Jul;248(1):224-33;

Zoubenko O V, Allison L A, Svab Z, Maliga P., 1994, Nucl. Acids. Res., 22, 3819-3824;

U.S. Pat. No. 5,693,507

WO9706250

WO9855595

WO9910513

WO9946394

WO0007431

1. A process for producing a multicellular plant or plant cells having stably transformed plastids comprising the following steps:
a) transforming plastids of said plant or plant cells via homologous recombination with at least one DNA molecule for enabling DNA modification, whereby said DNA molecule comprises a fragment of a gene of interest requiring for expression in a transformed plastid a sequence element of the host plastid not present in said DNA molecule;
b) subjecting said plastids to growth conditions which favour multiplication or allow for identification of plastids having said DNA modification; and
c) selecting or identifying plastids that are functional and contain information encoded in said DNA molecule;
thereby giving rise to functional cells or multicellular plants with stably transformed plastids.

2. The process according to claim 1, wherein said DNA molecule further comprises a mutated fragment of a plastid gene for creating a selection marker gene.

3. The process according to claim 2, wherein the DNA molecule is designed for a separation of the sites of insertion of the fagment of the gene of interest and the mutated fragment of a plastid gene on a plastome by independent events of homologous recombination.

4. The method of claim 2 or 3, wherein the mutation of said mutated fragment of a plastid gene is a point mutation.

5. The process of one of claims 1 to 4, wherein said fragment of a gene of interest requires for expression a plastome sequence element selected from the following group: a promoter sequence, a 5′-untranslated region, a start codon, a complete coding region, and a 3′-untranslated region or a portion thereof.

6. The process of one of claims 1 to 5, wherein said DNA modification comprises sequence replacement.

7. The process of one of claims 1 or 6, wherein said DNA modification comprises sequence insertion.

8. The process of one of claims 2 to 7, wherein said mutated fragment of a plastid gene is a mutated fragment of a plastid 23S rRNA gene, said mutated fragment conferring antibiotic resistance.

9. The process of one of claims 2 to 7, wherein said mutated fragment of a plastid gene is a mutated fragment of a plastid 16S rRNA gene, said mutated fragment conferring antibiotic resistance.

10. The process of one of claims 2 to 7, wherein said mutated fragment of a plastid gene is a mutated fragment of a plastid psbA gene, said mutated fragment conferring atrazine, metribuzin and/or diuron resistance.

11. The process of one of claims 2 to 7, wherein said mutated fragment of a plastid gene is a mutated fragment of a plastid atpB gene, said mutated fragment conferring tentoxin resistance.

12. The process of one of claims 1 to 11, wherein said DNA modification with a fragment of a gene of interest occurs between a 5′ regulatory sequence and an operably linked coding region and results in one or more additional cistrons.

13. The process of one of claims 1 to 11, wherein said DNA modification with a fragment of a gene of interest occurs between a coding region and an operably linked 3′ regulatory sequence and results in one or more additional cistrons.

14. The process of one of claims 1 to 11, wherein said DNA modification with a fragment of a gene of interest occurs directly downstream of a 3′ regulatory element.

15. The process according to one of claims 1 to 11, wherein said fragment of a gene of interest is a fragment of a heterologous gene, and wherein said DNA modification is designed for the change of a pre-existing cistron without creating an additional cistron by said change.

16. The process according to claim 15, wherein said changed cistron is designed for forming a hybrid messenger RNA comprising RNA sequence derived from host plastid DNA and RNA sequence derived from DNA of said fragment of a gene of interest.

17. The process according to claim 16, wherein said hybrid messenger RNA encodes one or multiple heterologous polypeptides or proteins.

18. The process according to one of claims 16 or 17, wherein translation of all or a part of said hybrid messenger RNA leads to a fusion protein.

19. The process according to claim 18, wherein said fusion protein comprises multiple heterologous polypeptide sequences.

20. The process according to one of claims 15 to 19, wherein said DNA molecule further comprises one or more sequence(s) each encoding a proteolytic cleavage.

21. The process according to claim 20, wherein said proteolytic cleavage site is autocatalytic.

22. The process according to one of claims 20 or 21, further comprising genetic or transient modification for providing a site-specific protease necessary for cleaving expressed fusion proteins having a proteolytic cleavage site.

23. The process according to one of claims 1 to 22, wherein DNA molecule further comprises one or more recombination sites or splicing sites.

24. The process of one of claims 1 to 15, wherein said stably transformed plastids of said functional cells or multicellular plants obtained by a first process of transformation are transformed with a second DNA molecule via homologous recombination for enabling DNA modification, whereby said second DNA molecule comprises a gene of interest or a fragment thereof requiring for expression in a transformed plastid a sequence element of the host plastid or of a fragment of a gene of interest introduced in said first process of transformation, optionally followed by the procedure defined in steps (b) and (c) of claim 1; and wherein said DNA modifications jointly generate an operon or a cistron.

25. The process according to claim 24, wherein the result of said DNA modifications is a functional transcriptionally active gene of interest.

26. The process according to claim 24, wherein said jointly generated operon or cistron is designed to form a hybrid messenger RNA comprising RNA derived from a fragment of a gene of interest of one DNA molecule and RNA derived from a second fragment of a gene of interest from said second DNA molecule.

27. The process according to claim 26, wherein at least a part of said hybrid messenger RNA is capable of being translated.

28. The process according to claim 26 or 27, wherein a functional protein is produced comprising a polypeptide encoded by a first fragment of a gene of interest of a first DNA molecule and a polypeptide encoded by the second fragment of a gene of interest from the second DNA molecule.

29 The process of one of claims 1 to 28 yielding plastids that are at least partially functional as plastids and express additionally one or more useful traits.

30. The process according to one of claims 1 to 29, wherein said DNA modification with a fragment of a gene of interest is effected in a transcribed region of the genome of said plastids.

31. The process of one of claims 4 to 28, wherein said process is carried out twice and wherein a point mutation created in the first run is reverted in the second run while creating another point mutation in the same or in another plastid gene.

32. The process of one of claims 1 to 31, wherein said DNA modification comprises insertion of a gene of interest into the plastome of said plastids.

33. The process of one of claims 1 to 32, wherein said DNA modification comprises sequence deletion.

34. Transformed plastids, cells or multicellular plants and their progeny, obtained using the process of one of claims 1 to 33.

35. Chimeric transformed cells or multicellular plants and their progeny, containing a mixture of wild type and transformed plastids, obtained using the process of one of claims 1 to 33.

36. Stable chimeric plants, containing a mixture of at least two differently transformed plastids, obtained using the process of one of claims 1 to 33.

37. DNA molecule for performing the process according to one of claims 1 to 33.


SEQUENCE LISTING (53 sequences)

SEQ ID NO: 1 - DNA, 25 bp, Artificial Sequence
  PCR Primer
tgaattcccatggctcgtgaagcgg

SEQ ID NO: 2 - DNA, 32 bp, Artificial Sequence
  PCR Primer
tatggatccttgccaactaccttagtgatctc

SEQ ID NO: 3 - DNA, 36 bp, Artificial Sequence
  PCR Primer
acaagagctcataagtaataaaacgttcgaataatt

SEQ ID NO: 4 - DNA, 30 bp, Artificial Sequence
  PCR Primer
aattcctcgagtaggtcgatggggaaaatg

SEQ ID NO: 5 - DNA, 34 bp, Artificial Sequence
  PCR Primer
tatagggcccagctataggtttacatttttaccc

SEQ ID NO: 6 - DNA, 31 bp, Artificial Sequence
  PCR Primer
catgctgcagcaagaaaataacctctccttc

SEQ ID NO: 7 - DNA, 30 bp, Artificial Sequence
  PCR Primer
tttcctgcagttattcatgattgagtattc

SEQ ID NO: 8 - DNA, 21 bp, Artificial Sequence
  PCR Primer
ccagaaagaagtatgctttgg

SEQ ID NO: 9 - DNA, 28 bp, Artificial Sequence
  PCR Primer
ggatccatgcgtgaagcggttatcgccg

SEQ ID NO: 10 - DNA, 32 bp, Artificial Sequence
  PCR Primer
ctgggtaccttattgtttgcctccctgctgcg

SEQ ID NO: 11 - DNA, 24 bp, Artificial Sequence
  PCR Primer
catgccatggtccgtcctgtagaa

SEQ ID NO: 12 - DNA, 37 bp, Artificial Sequence
  PCR Primer
ggggtaccagttgtagggagggatccatgcgtgaagc

SEQ ID NO: 13 - DNA, 37 bp, Artificial Sequence
  PCR Primer
ctataccatggtgcttttcaaatcctcctagcctgca

SEQ ID NO: 14 - DNA, 71 bp, Artificial Sequence
  PCR Primer
ctataccatggggtagaacctcctcagggaatataaggttttgatgaggctgatcttgagccgccactgca

SEQ ID NO: 15 - DNA, 60 bp, Artificial Sequence
  PCR Primer
ctataccatggtttgcctcctactactgaatcataagcatgtagattttttttatctgca

SEQ ID NO: 16 - DNA, 32 bp, Artificial Sequence
  PCR Primer
catgctgcagttatgaaatcggattgatagcc

SEQ ID NO: 17 - DNA, 29 bp, Artificial Sequence
  PCR Primer
tatagggcccgaagtatcggccttattgg

SEQ ID NO: 18 - DNA, 34 bp, Artificial Sequence
  PCR Primer
catgctgcagttggtacgttcgaataataaaaag

SEQ ID NO: 19 - DNA, 34 bp, Artificial Sequence
  PCR Primer
tatagaagcttgcattgggctctttcattaactg

SEQ ID NO: 20 - DNA, 20 bp, Artificial Sequence
  PCR Primer
gctggcggcatgcttaacac

SEQ ID NO: 21 - DNA, 23 bp, Artificial Sequence
  PCR Primer
ccagcatgcattagctctccctg

SEQ ID NO: 22 - DNA, 30 bp, Artificial Sequence
  PCR Primer
cctctagagggtattttggtttgacactgc

SEQ ID NO: 23 - DNA, 30 bp, Artificial Sequence
  PCR Primer
agctgcagtccaaccaattgggagagaatc

SEQ ID NO: 24 - DNA, 21 bp, Artificial Sequence
  PCR Primer
tccggaatgattgggcgtaaa

SEQ ID NO: 25 - DNA, 20 bp, Artificial Sequence
  PCR Primer
ggcggtgtgtacaaggcccg

SEQ ID NO: 26 - DNA, 20 bp, Artificial Sequence
  PCR Primer
agtaggagtggaaggaggcc

SEQ ID NO: 27 - DNA, 20 bp, Artificial Sequence
  PCR Primer
acagttcagtagtacgggga

SEQ ID NO: 28 - DNA, 20 bp, Artificial Sequence
  PCR Primer
tcaaagtaagaacgcttgca

SEQ ID NO: 29 - DNA, 20 bp, Artificial Sequence
  PCR Primer
ttttccttaactgcccccgg

SEQ ID NO: 30 - DNA, 28 bp, Artificial Sequence
  PCR Primer
aggcatgcaaacttctgtcgctccatcc

SEQ ID NO: 31 - DNA, 27 bp, Artificial Sequence
  PCR Primer
aggcatgctaagatcaggccgaaaggc

SEQ ID NO: 32 - DNA, 35 bp, Artificial Sequence
  PCR Primer
gtccatcaggcgtaaaagtgtctgtacagataaag

SEQ ID NO: 33 - DNA, 30 bp, Artificial Sequence
  PCR Primer
gtggacctgtccctctgggatacttcgaag

SEQ ID NO: 34 - DNA, 35 bp, Artificial Sequence
  PCR Primer
gggaattccatatgagaatcaatcctactacttct

SEQ ID NO: 35 - DNA, 32 bp, Artificial Sequence
  PCR Primer
aaaactgcagaatcgtctgcgggtacataaac

SEQ ID NO: 36 - DNA, 30 bp, Artificial Sequence
  PCR Primer
agtcgagctcatgtaccagtagaagattcg

SEQ ID NO: 37 - DNA, 36 bp, Artificial Sequence
  PCR Primer
cgggatccaataataaaataaataaatatgtcgaaa

SEQ ID NO: 38 - DNA, 35 bp, Artificial Sequence
  PCR Primer
tactttcaacaactcgcgatcgttacacttcttcc

SEQ ID NO: 39 - DNA, 31 bp, Artificial Sequence
  PCR Primer
catctagactgctttgccaagggtaccaatg

SEQ ID NO: 40 - DNA, 31 bp, Artificial Sequence
  PCR Primer
gaccatggaaccttcaatggtgagatgaaac

SEQ ID NO: 41 - DNA, 28 bp, Artificial Sequence
  PCR Primer
gaggatcctggagatgttttgcttaacg

SEQ ID NO: 42 - DNA, 29 bp, Artificial Sequence
  PCR Primer
gatctagacaattatggacgacaacctgg

SEQ ID NO: 43 - DNA, 21 bp, Artificial Sequence
  PCR Primer
atgccatggctcgtgaagcgg

SEQ ID NO: 44 - DNA, 26 bp, Artificial Sequence
  PCR Primer
gaggatccttgccaactaccttagtg

SEQ ID NO: 45 - DNA, 25 bp, Artificial Sequence
  PCR Primer
caggatccactggccgtcgttttac

SEQ ID NO: 46 - DNA, 28 bp, Artificial Sequence
  PCR Primer
gatgaagcttggtcatagctgtttcctg

SEQ ID NO: 47 - DNA, 28 bp, Artificial Sequence
  PCR Primer
cgaagcttttttctttttattaagttca

SEQ ID NO: 48 - DNA, 34 bp, Artificial Sequence
  PCR Primer
catatgcctgcaggtatataataattcagagaaa

SEQ ID NO: 49 - DNA, 27 bp, Artificial Sequence
  PCR Primer
gtctagaagatacccatgtatatcttg

SEQ ID NO: 50 - DNA, 28 bp, Artificial Sequence
  PCR Primer
gcccgggagttaaaatacctgacgtagc

SEQ ID NO: 51 - DNA, 29 bp, Artificial Sequence
  PCR Primer
gcctgcagggccgtcgttcaatgagaatg

SEQ ID NO: 52 - DNA, 35 bp, Artificial Sequence
  PCR Primer
gcccgggttaattaatcattgtttgcctccctgct

SEQ ID NO: 53 - DNA, 42 bp, Artificial Sequence
  Ligation Oligo
ttaattaatggctaggaggatttgaaaagcattcatatgccg